(19) United States
(12) Patent Application Publication
(10) Pub. No.: US 2026/0110032 A1
(43) Pub. Date: Apr. 23, 2026

(54) METHODS FOR DETERMINATION AND MONITORING OF XENOTRANSPLANT REJECTION BY MEASURING NUCLEIC ACIDS OR PROTEINS DERIVED FROM THE XENOTRANSPLANT

(71) Applicant: Natera, Inc., San Carlos, CA (US)

(72) Inventors: Tudor Pompiliu CONSTANTIN, Berkeley, CA (US); Bernhard ZIMMERMANN, Manteca, CA (US); Gabrielle HEILEK, San Carlos, CA (US); Swee Seong WONG, Carmel, IN (US)

(73) Assignee: Natera, Inc., San Carlos, CA (US)

(21) Appl. No.: 19/117,608

(22) PCT Filed: Sep. 27, 2023
(86) PCT No.: PCT/US2023/033874

§ 371 Date: Apr. 1, 2025

Related U.S. Application Data
(60) Provisional application No. 63/413,738, filed on Oct. 6, 2022.

(51) Int. Cl. C12Q 1/6883; C12N 9/22; C12N 15/10; C12N 15/11; C12Q 1/37; C12Q 1/6806; C12Q 1/6809; C12Q 1/6848; C12Q 1/6874

(52) U.S. Cl. CPC C12Q 1/6883; C12N 9/226; C12N 15/1096; C12N 15/111; C12Q 1/37; C12Q 1/6806; C12Q 1/6809; C12Q 1/6848; C12Q 1/6874; C12N 2310/20; C12Q 2600/106; G01N 2333/922

(57) ABSTRACT

The present disclosure provides methods for preparation and analysis of biological samples of xenotransplant recipients, wherein the methods comprise extracting fragmented or intact cell-free DNA, RNA (such as mRNA or miRNA), or protein derived from sample of the xenotransplant recipient, and measuring amounts of cell-free DNA, RNA (such as mRNA or miRNA), or protein derived from the xenotransplant enables assessment of xenotransplant rejection. The detection of the cell-free DNA, or RNA may be performed by preparing sequencing libraries and performing whole genome sequencing.

### CROSS-REFERENCE TO RELATED APPLICATION

This application claims priority to and the benefit of U.S. Provisional Patent Application No. 63/413,738, filed Oct. 6, 2022, which is incorporated herein by reference in its entirety.

### BACKGROUND

Rapid detection of graft injury and/or rejection remains a challenge for transplant recipients. Xenotransplantation, where animal organs are transplanted into human patients with terminal organ failure, is a potential approach to alleviating the chronic shortage of transplantable organs. Several efforts are under way to bring this approach into the clinic, including the production of transgenic donor animal strains with reduced immunogenicity and with low risk of passing animal viruses to humans, as well as clinical tests where animal organs are transplanted into human patients. The rapid progress of this research and the severe shortage of human-origin organs for transplant make it likely that xenotransplantation can become the leading approach for addressing terminal organ failure in the clinic in the near future.

Following xenotransplantation, tests for determining and monitoring transplant injury and/or rejection is a critical aspect of the post-transplantation care and determination of need for personalized immunosuppressive therapy. Conventional biopsy-based tests are invasive and costly and possibly lead to late diagnosis of transplant injury and/or rejection. Therefore, there is a need for a non-invasive test for xenotransplant injury and/or rejection that is more sensitive and more specific than conventional biopsy-based tests to enable early diagnosis of transplant injury and/or rejection.

Thus, improved methods are needed to accurately diagnose, screen, test, and monitor xenotransplant injury and/or rejection at an early stage. The present disclosure provides this need.

### SUMMARY

In one aspect, the present disclosure relates to a method of preparing a composition of DNA derived from a blood, plasma, serum or urine sample of a xenotransplant recipient useful for determination of xenotransplant rejection, comprising: (a) extracting cell-free DNA from the blood, plasma, serum or urine sample of the xenotransplant recipient, wherein the extracted cell-free DNA comprises donor-derived cell-free DNA and recipient-derived cell-free DNA; (b) preparing a sequencing library from the extracted cell-free DNA, and sequencing the sequencing library by high-throughput sequencing to obtain sequencing reads and quantifying the total amount of animal donor-derived cell-free DNA based on the sequencing reads; (c) determining whether the amount of donor-derived cell-free DNA from the xenotransplant or a function thereof exceeds a cutoff threshold indicating xenotransplant rejection or injury.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, scrum or urine sample of the xenotransplant recipient, wherein the extracted cell-free DNA comprises donor-derived cell-free DNA from the xenotransplant and recipient-derived cell-free DNA; (b) preparing a composition of amplified DNA by performing multiplex targeted amplification of the extracted cell-free DNA at 10-50,000 target loci in a single reaction volume to detect and quantify the amount of animal donor-derived cell-free DNA, wherein the target loci comprise a set of animal target loci and a set of human target loci; (c) determining whether the amount of animal donor-derived cell-free DNA or a function thereof exceeds a cutoff threshold indicating xenotransplant rejection.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, serum or urine sample of the transplant recipient, wherein the extracted cell-free DNA comprises donor-derived cell-free DNA and recipient-derived cell-free DNA; (b) preparing a composition of amplified DNA by performing targeted amplification of the extracted cell-free DNA at 10-50,000 target loci in a single reaction volume, wherein the target loci comprise both human and animal target loci, and sequencing the amplified DNA by high-throughput sequencing to obtain sequencing reads and quantifying the amount of both donor-derived cell-free DNA and xenotransplant recipient-derived cell-free DNA based on the sequencing reads, wherein the human and animal loci are the same, and wherein the human and animal reads are distinguished based on an insert sequence; (c) determining whether the fraction of animal donor-derived cell-free DNA or a function thereof exceeds a cutoff threshold indicating xenotransplant rejection.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, serum or urine sample of the xenotransplant recipient, wherein the extracted cell-free DNA comprises donor-derived cell-free DNA and recipient-derived cell-free DNA; (b) preparing a composition of amplified DNA by performing targeted amplification of the extracted cell-free DNA at 10-50,000 target loci in a single reaction volume, wherein the target loci comprise one or more target loci indicating xenotransplant rejection; and (c) determining an amount of the one or more target loci indicating xenotransplant rejection, and determining whether the amount of the one or more target loci indicating xenotransplant rejection or a function thereof exceeds a cutoff threshold indicating xenotransplant rejection.

In one aspect, the present disclosure relates to a method of preparing a composition of DNA derived from a blood, plasma, serum or urine sample of a xenotransplant recipient useful for determination of xenotransplant rejection, wherein the composition of DNA comprises one or more target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection; wherein xenotransplant rejection is determined by a combination of (i) the amount of the one or more target loci indicating xenotransplant rejection or a function thereof, and (ii) the total amount of animal donor-derived cell-free DNA or the fraction of animal donor-derived cell-free DNA.

In one In one aspect, the disclosure herein relates to a method of preparing a composition of complimentary DNA (cDNA) from RNA extracted from a blood, plasma, serum or urine sample of a xenotransplant recipient useful for determination of xenotransplant rejection, comprising: (a) extracting RNA from the blood, plasma, scrum or urine sample of the xenotransplant recipient, wherein the extracted RNA comprises donor-derived RNA and recipient-derived RNA; (b) preparing a sequencing library of cDNA from the extracted RNA, and sequencing the sequencing library of cDNA by high-throughput sequencing to obtain sequencing reads and quantifying the total amount of donor-derived RNA based on the sequencing reads; (c) determining whether the total amount of donor-derived RNA from the xenotransplant or a function thereof exceeds a cutoff threshold indicating xenotransplant rejection.

In one aspect, the disclosure herein relates to a method of preparing a composition of amplified complimentary DNA (cDNA) from RNA extracted from a blood, plasma, serum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived RNA from the xenotransplant and recipient-derived RNA; (b) preparing a composition of amplified cDNA derived from the extracted RNA by performing multiplex targeted amplification of the cDNA at 10-50,000 target loci in a single reaction volume to detect and quantify the amount of donor-derived RNA, wherein the target loci comprise a set of animal target loci and a set of human target loci; (c) determining whether the amount of donor-derived RNA target loci or a function thereof exceeds a cutoff threshold indicating xenotransplant rejection.

In one aspect, the disclosure herein relates to a method of preparing a composition of amplified complimentary DNA (cDNA) from RNA extracted from a blood, plasma, serum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived RNA and recipient-derived RNA; (b) preparing a composition of amplified cDNA derived from the extracted RNA by performing targeted amplification of the cDNA at 10-50,000 target loci in a single reaction volume, wherein the target loci comprise both human and target loci, and sequencing the amplified cDNA by high-throughput sequencing to obtain sequencing reads and quantifying the amount of both donor-derived RNA and xenotransplant recipient-derived RNA based on the sequencing reads, wherein the human and animal loci are the same, and wherein the human and animal reads are distinguished based on an insert sequence; (c) determining whether the fraction of donor-derived RNA or a function thereof exceeds a cutoff threshold indicating xenotransplant rejection.

In one aspect, the disclosure herein relates to a method of preparing a composition of amplified complimentary DNA (cDNA) from RNA extracted amplified complimentary DNA (cDNA) from RNA extracted from a blood, plasma, serum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived cell-free RNA and recipient-derived RNA; (b) preparing a composition of amplified cDNA derived from the extracted RNA by performing targeted amplification of the cDNA at 10-50,000 target loci in a single reaction volume, wherein the target loci comprise one or more target loci indicating xenotransplant rejection; and (c) determining an amount of the one or more target loci indicating xenotransplant rejection, and whether the amount of the one or more target loci indicating xenotransplant rejection or a function thereof exceeds a cutoff threshold indicating xenotransplant rejection.

In one aspect, the disclosure herein relates to a method of preparing a composition of amplified complimentary DNA (cDNA) from RNA extracted amplified complimentary DNA (cDNA) from RNA extracted from a blood, plasma, scrum or urine sample of a xenotransplant recipient useful for determination of xenotransplant rejection, wherein the composition comprises target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection, wherein xenotransplant rejection is determined by a combination of the amount of the one or more target loci indicating xenotransplant rejection or a function thereof, and the total amount of animal donor-derived RNA or the fraction of animal donor-derived RNA.

In one aspect the present disclosure relates to a method of preparing a composition of protein derived from a blood, plasma, serum or urine sample of a xenotransplant recipient useful for determination of xenotransplant rejection, comprising: (a) extracting protein from the blood, plasma, scrum or urine sample of the xenotransplant recipient, wherein the protein comprises donor-derived protein from the xenotransplant and recipient-derived protein; (b) detecting and quantifying an amount of donor-derived protein; (c) determining whether the amount of protein donor-derived protein or a function thereof exceeds a cutoff threshold indicating xenotransplant rejection.

In one aspect the present disclosure relates to a method of preparing a composition of protein derived from a blood, plasma, serum or urine sample of a xenotransplant recipient useful for determination of xenotransplant rejection, comprising: (a) extracting protein from the blood, plasma, serum or urine sample of the xenotransplant recipient, wherein the protein comprises donor-derived protein from the xenotransplant and recipient-derived protein; (b) detecting and quantifying an amount of one or more target proteins, wherein the one or more target proteins comprise both recipient-derived proteins and donor-derived proteins, and wherein the one or more target proteins are indicative of xenotransplant rejection; (c) determining whether the amount of the one or more target proteins or a function thereof exceeds a cutoff threshold indicating xenotransplant rejection.

In one aspect the present disclosure relates to a method of administrating immunosuppressive therapy in a xenotransplant recipient, comprising: (a) measuring an amount of donor-derived protein as described herein; and (b) titrating the dosage of an immunosuppressive therapy according to the amount of donor-derived protein or a function thereof.

In one aspect the present disclosure relates to a method of administrating immunosuppressive therapy in a xenotransplant recipient, comprising: (a) measuring an amount of one or more target proteins as described herein; and (b) titrating the dosage of an immunosuppressive therapy according to the amount of the one or more target proteins or a function thereof.

In some embodiments, the method herein further comprises repeating steps (a)-(b) longitudinally for the same xenotransplant recipient, and determining a longitudinal change in the amount donor-derived protein, donor-derived target proteins, or a function thereof, and a longitudinal change in the amount of donor-derived protein, target proteins, or a function thereof.

In some embodiments, the method herein further comprises titrating the dosage of the immunosuppressive therapy according to the longitudinal change in the donor-derived protein, donor-derived target proteins, or a function thereof.

In some embodiments, the protein is derived from extracellular vehicles (EVs) isolated from the blood, plasma, serum, or urine samples of a xenotransplant recipient, and comprises extracting protein from extracellular vesicles (EVs) isolated from the blood, plasma, serum or urine sample of the xenotransplant recipient.

In one aspect the present disclosure relates to a method of administrating immunosuppressive therapy in a xenotransplant recipient, comprising: (a) measuring the amount of cell-free DNA in a blood, plasma, serum, or urine sample of the transplant recipient; and (b) measuring the total amount of donor-derived cell-free DNA in a blood, plasma, serum, or urine sample of the transplant recipient; and (c) titrating the dosage of an immunosuppressive therapy according to the amount of cell-free DNA or a function thereof and the amount of donor-derived cell-free DNA or a function thereof.

In some embodiments, the amount of donor-derived cell-free DNA is measured by: extracting cell-free DNA from the blood, plasma, serum or urine sample of the transplant recipient, wherein the extracted cell-free DNA comprises donor-derived cell-free DNA and recipient-derived cell-free DNA; performing targeted amplification of the extracted DNA at 200-50,000 target loci in a single reaction volume; sequencing the amplified DNA by high-throughput sequencing to obtain sequencing reads and quantifying the amount of donor-derived cell-free DNA based on the sequencing reads.

In some embodiments, the method herein further comprises repeating steps (a)-(b) longitudinally for the same transplant recipient, and determining a longitudinal change in the amount of cell-free DNA or a function thereof and a longitudinal change in the amount of donor-derived cell-free DNA or a function thereof.

In some embodiments, the method herein further comprises titrating the dosage of the immunosuppressive therapy according to the longitudinal change in the total amount of cell-free DNA or a function thereof and the longitudinal change in the amount of donor-derived cell-free DNA or a function thereof.

In one aspect the present disclosure relates to a method of administrating immunosuppressive therapy in a xentransplant recipient, comprising: (a) measuring the amount of RNA in a blood, plasma, serum, or urine sample of the transplant recipient; (b) measuring the amount of donor-derived RNA in a blood, plasma, serum, or urine sample of the transplant recipient; and (c) titrating the dosage of an immunosuppressive therapy according to the amount of cell-free DNA or a function thereof and the amount of donor-derived RNA or a function thereof.

In some embodiments, the amount of donor-derived RNA is measured by: extracting RNA from the blood, plasma, serum or urine sample of the transplant recipient, wherein the extracted RNA comprises donor-derived RNA and recipient-derived RNA; preparing a composition of amplified complimentary DNA (cDNA) derived from the extracted RNA by performing multiplex targeted amplification of the cDNA at 200-50,000 animal target loci in a single reaction volume to detect and quantify the amount of animal donor-derived RNA; sequencing the amplified cDNA by high-throughput sequencing to obtain sequencing reads and quantifying the amount of donor-derived RNA based on the sequencing reads.

In some embodiments, the method herein further comprises repeating steps (a)-(b) longitudinally for the same transplant recipient, and determining a longitudinal change in the amount of RNA or a function thereof and a longitudinal change in the amount of donor-derived RNA or a function thereof.

In some embodiments, the method herein further comprises titrating the dosage of the immunosuppressive therapy according to the longitudinal change in the total amount of RNA or a function thereof and the longitudinal change in the amount of donor-derived cell-free DNA or a function thereof.

In some embodiments, an internal control is added to the sample.

In some embodiments, the sequencing comprises shotgun whole genome sequencing.

In some embodiments, the amount of DNA or RNA is measured by quantitative PCR, real-time PCR, digital PCR, or sequencing.

In some embodiments, sequencing comprises next-generation whole genome sequencing.

In some embodiments, wherein the amount of RNA or cell-free DNA is measured by using microarray.

In some embodiments, the amount of donor-derived RNA or cell-free DNA is determined by using ratiometric and/or machine learning-artificial intelligence comparisons at a single or a plurality of time points.

In some embodiments, the amount of RNA or cell-free DNA is measured by using molecular barcodes and microscopic imaging (such as NanoString NCOUNTER®).

In some embodiments, the target loci comprise single nucleotide polymorphisms (SNPs).

In some embodiments, the cutoff threshold is an estimated percentage of donor-derived cell-free DNA or RNA out of total cell-free DNA or RNA or a function thereof.

In some embodiments, the cutoff threshold is proportional to an absolute donor-derived cell-free DNA concentration or RNA concentration.

In some embodiments, step (b) comprises amplification of at least 2, at least 5, at least 10, at least 20, at least 30, at least 50, at least 100 target loci, from 2-10, 200-100, 50-500, or 50-2000 target loci, using at least at least 2, at least 5, at least 10, at least 20, at least 30, at least 50, at least 100 target RNA molecules, from 2-10, 200-100, 50-500, or 50-2000 pairs of forward and reverse PCR primers.

In some embodiments, step (b) comprises multiplex amplification of at least 100, at least 500, at least 1000, at least 2000 target loci, from 10-1000, 100-10000, 50-50000, or 500-20000 target loci, using at least 100, at least 500, at least 1000, at least 2000, from 10-1000, 100-10000, 50-50000, 500-20000 pairs of forward and reverse PCR primers.

In some embodiments, the RNA is cell-free RNA.

In some embodiments, the cell-free RNA is derived from exosomes or microvesicles.

In some embodiments, the RNA is small messenger RNA (mRNA).

In some embodiments, wherein the RNA is small noncoding RNA (sncRNA).

In some embodiments, the sncRNA comprises micro RNA (miRNA), piwi-interacting RNA (piRNA), small nucleolar RNA (snoRNA), small nuclear RNA (snRNA), or miscellaneous RNA (miscRNA).

In some embodiments, the methods herein further comprises utilizing CRISPR-Cas to target and deplete contaminating or overabundant nucleic acid species in the sample, thereby increasing the fraction of desired reads that map to a target loci of interest per sample and the sample throughput per sequencing run

In some embodiments, the sample comprises whole blood or hemolysis tainted blood, serum or plasma samples, and wherein a plurality of guide RNAs are used to target a plurality of loci in the same reaction to deplete contaminating or overabundant nucleic acid, thereby increasing a detection rate of the target loci.

In some embodiments, the contaminating or overabundant nucleic acid species comprise hemoglobin mRNA, tRNA, and rRNA, and miR-451, miR-144, and miR-486.

In some embodiments, the method herein further comprises depleting adaptor dimers, primer dimers, unwanted ligation products from the composition of amplified nucleic acids comprising target loci, thereby increasing the fraction of desired reads that map to a target loci of interest per sample and the sample throughput per sequencing run.

In some embodiments, Cas9/Cas12a is utilized to remove nucleic acid species after reverse transcription of RNA and before multiplex amplification.

In some embodiments, Cas9/Cas12a is utilized to remove nucleic acid species after 1-10 cycles of multiplex amplification of the complementary DNA.

In some embodiments, the contaminating or overabundant nucleic acid species are RNA, and wherein Cas13 is used to remove the contaminating or overabundant RNA species from the sample.

In some embodiments, the xenotransplant recipient is a human subject.

In some embodiments, the xenotransplant recipient has received one or more xenotransplanted organs selected from pancreas, kidney, liver, heart, lung, intestinal, thymus, and uterus.

In some embodiments, the sample is obtained from the xenotransplant recipient less than 18 months post-transplantation.

In some embodiments, the rejection risk for the xenotransplant recipient is determined using logistic regression, random forest, or decision tree machine learning analysis.

In some embodiments, the logistic regression, random forest, or decision tree machine learning analysis further incorporates one or more parameters selected from time post-transplantation, age of xenotransplant recipient and/or xenotransplant donor, gender of xenotransplant recipient and/or xenotransplant donor.

In some embodiments, the xenotransplant is from a pig, a primate, a baboon, a cow, or a dog.

In some embodiments, the cell-free DNA or RNA is derived from extracellular vehicles (EVs) isolated from the blood, plasma, serum, or urine samples of a xenotransplant recipient.

### BRIEF DESCRIPTION OF THE DRAWINGS

FIG. 1. Workflow for testing methods of detecting cell-free DNA from animal donor in human plasma sample by using a whole genome sequencing approach.

FIG. 2. Graph depicting detecting cell-free DNA from animal donor in human plasma sample. The graph shows linear relationship between known amount of added animal derived cell-free DNA, and the amount measured by using whole genome sequencing, thereby demonstrating proof of concept of the methods of using whole genome sequencing to measure animal donor derived cell-free DNA in human plasma samples.

### DETAILED DESCRIPTION

The present disclosure relates to methods of determining and monitoring xenotransplant rejection in a human recipient based on using whole genome sequencing of cell-free DNA or RNA of blood, plasma, serum or urine sample of the xenotransplant recipient. Alternatively, the present disclosure relates to methods of determining and monitoring xenotransplant rejection in a human recipient based on measuring protein derived from blood, plasma, serum or urine sample of a xenotransplant recipient. In some embodiments, the cell-DNA, RNA, or protein is isolated from extracellular vehicles (EVs) isolated from blood, plasma, serum or urine sample of a xenotransplant recipient. The working examples presented herein show that the presently disclosed methods can be used to detect nucleic acids derived from an animal in a human plasma sample.

### Methods of Determining and Monitoring Xenotransplant Rejection Based on Measuring Cell-Free DNA

In one aspect, the present disclosure relates to a method of preparing a composition of DNA derived from a blood, plasma, serum or urine sample of a xenotransplant recipient useful for determination of xenotransplant rejection, comprising: (a) extracting cell-free DNA from the blood, plasma, serum or urine sample of the xenotransplant recipient, wherein the extracted cell-free DNA comprises donor-derived cell-free DNA and recipient-derived cell-free DNA; (b) preparing a sequencing library from the extracted cell-free DNA, and sequencing the sequencing library by high-throughput sequencing to obtain sequencing reads and quantifying the total amount of animal donor-derived cell-free DNA based on the sequencing reads; (c) determining whether the amount of donor-derived cell-free DNA from the xenotransplant or a function thereof exceeds a cutoff threshold indicating xenotransplant rejection or injury. In some embodiments, no amplification or pre-amplification is performed on the extracted cell-free DNA prior to sequencing. In some embodiments, preparing a sequencing library comprises attaching adapters to the extracted cell-free DNA for example by ligation. In some embodiments, attaching adapters to the extracted cell-free DNA comprises end repair, addition of an adenosine to the cell-free DNA fragments, followed by sticky end ligation to the cell-free DNA fragments. In some embodiments, the cell-free DNA fragments are repaired and filled to generated blunt end. In some embodiments, attaching adapters to the extracted cell-free DNA comprises blunt end ligation of adapters to the cell-free DNA fragments.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, serum or urine sample of the xenotransplant recipient, wherein the extracted cell-free DNA comprises donor-derived cell-free DNA from the xenotransplant and recipient-derived cell-free DNA; (b) preparing a composition of amplified DNA by performing multiplex targeted amplification of the extracted cell-free DNA at 10-50,000 target loci in a single reaction volume to detect and quantify the amount of animal donor-derived cell-free DNA, wherein the target loci comprise a set of animal target loci and a set of human target loci; (c) determining whether the amount of animal donor-derived cell-free DNA or a function thereof exceeds a cutoff threshold indicating xenotransplant rejection.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, serum or urine sample of the transplant recipient, wherein the extracted cell-free DNA comprises donor-derived cell-free DNA and recipient-derived cell-free DNA; (b) preparing a composition of amplified DNA by performing targeted amplification of the extracted cell-free DNA at 10-50,000 target loci in a single reaction volume, wherein the target loci comprise both human and animal target loci, and sequencing the amplified DNA by high-throughput sequencing to obtain sequencing reads and quantifying the amount of both donor-derived cell-free DNA and xenotransplant recipient-derived cell-free DNA based on the sequencing reads, wherein the human and animal loci are the same, and wherein the human and animal reads are distinguished based on an insert sequence; (c) determining whether the fraction of animal donor-derived cell-free DNA or a function thereof exceeds a cutoff threshold indicating xenotransplant rejection. As used herein, “an insert sequence” refers to any sequence that is different in the host human target loci compared to the same animal target loci.

In one aspect, the present disclosure relates to a method of preparing a composition of amplified DNA derived from a blood, plasma, serum or urine sample of a xenotransplant recipient useful for determination of xenotransplant rejection, comprising: (a) extracting cell-free DNA from the blood, plasma, serum or urine sample of the xenotransplant recipient, wherein the extracted cell-free DNA comprises donor-derived cell-free DNA and recipient-derived cell-free DNA; (b) preparing a composition of amplified DNA by performing targeted amplification of the extracted cell-free DNA at 10-50,000 target loci in a single reaction volume, wherein the target loci comprise one or more target loci indicating xenotransplant rejection; and (c) determining an amount of the one or more target loci indicating xenotransplant rejection, and determining whether the amount of the one or more target loci indicating xenotransplant rejection or a function thereof exceeds a cutoff threshold indicating xenotransplant rejection.

In one aspect, the present disclosure relates to a method of preparing a composition of DNA derived from a blood, plasma, serum or urine sample of a xenotransplant recipient useful for determination of xenotransplant rejection, wherein the composition of DNA comprises one or more target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection; wherein xenotransplant rejection is determined by a combination of (i) the amount of the one or more target loci indicating xenotransplant rejection or a function thereof, and (ii) the total amount of animal donor-derived cell-free DNA or the fraction of animal donor-derived cell-free DNA.

In some embodiments, the cell-free DNA is derived from extracellular vesicles (EVs) isolated from a blood, plasma, serum or urine samples of a xenotransplant recipient.

### Methods of Determining and Monitoring Xenotransplant Rejection Based on Measuring RNA

In one aspect, the disclosure herein relates to a method of preparing a composition of complimentary DNA (cDNA) from RNA extracted from a blood, plasma, scrum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived RNA and recipient-derived RNA; (b) preparing a sequencing library of cDNA from the extracted RNA, and sequencing the cDNA by high-throughput sequencing to obtain sequencing reads and quantifying the total amount of donor-derived RNA based on the sequencing reads; (c) determining whether the total amount of donor-derived RNA from the xenotransplant or a function thereof exceeds a cutoff threshold indicating xenotransplant rejection. In some embodiments, no amplification or pre-amplification is performed on the extracted RNA prior to sequencing. In some embodiments, preparing a sequencing library comprises attaching adapters to the cDNA for example by ligation. In some embodiments, the cDNA fragments are repaired and filled to generated blunt ends. In some embodiments, adapters are appended to the cDNA fragments by blunt end ligation. In some embodiments, the cDNA are appended with adapters by sticky end ligation to generate a sequencing library of cDNA.

In one aspect, the disclosure herein relates to a method of preparing a composition of amplified complimentary DNA (cDNA) from RNA extracted from a blood, plasma, serum or urine sample of a xenotransplant recipient useful for determination of xenotransplant rejection, comprising: (a) extracting RNA from the blood, plasma, scrum or urine sample of the xenotransplant recipient, wherein the extracted RNA comprises donor-derived RNA from the xenotransplant and recipient-derived RNA; (b) preparing a composition of amplified cDNA derived from the extracted RNA by performing multiplex targeted amplification of the cDNA at 10-50,000 target loci in a single reaction volume to detect and quantify the amount of donor-derived RNA, wherein the target loci comprise a set of animal target loci and a set of human target loci; (c) determining whether the amount of donor-derived RNA target loci or a function thereof exceeds a cutoff threshold indicating xenotransplant rejection.

In one aspect, the disclosure herein relates to a method of preparing a composition of amplified complimentary DNA (cDNA) from RNA extracted from a blood, plasma, serum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived RNA and recipient-derived RNA; (b) preparing a composition of amplified cDNA derived from the extracted RNA by performing targeted amplification of the cDNA at 10-50,000 target loci in a single reaction volume, wherein the target loci comprise both human and target loci, and sequencing the amplified cDNA by high-throughput sequencing to obtain sequencing reads and quantifying the amount of both donor-derived RNA and xenotransplant recipient-derived RNA based on the sequencing reads, wherein the human and animal loci are the same, and wherein the human and animal reads are distinguished based on an insert sequence; (c) determining whether the fraction of donor-derived RNA or a function thereof exceeds a cutoff threshold indicating xenotransplant rejection.

In one aspect, the disclosure herein relates to a method of preparing a composition of amplified complimentary DNA (cDNA) from RNA extracted amplified complimentary DNA (cDNA) from RNA extracted from a blood, plasma, serum or urine sample of a xenotransplant recipient useful for determination of xenotransplant rejection, comprising: (a) extracting RNA from the blood, plasma, serum or urine sample of the xenotransplant recipient, wherein the extracted RNA comprises donor-derived cell-free RNA and recipient-derived RNA; (b) preparing a composition of amplified cDNA derived from the extracted RNA by performing targeted amplification of the cDNA at 10-50,000 target loci in a single reaction volume, wherein the target loci comprise one or more target loci indicating xenotransplant rejection; and (c) determining an amount of the one or more target loci indicating xenotransplant rejection, and whether the amount of the one or more target loci indicating xenotransplant rejection or a function thereof exceeds a cutoff threshold indicating xenotransplant rejection.

In one aspect, the disclosure herein relates to a method of preparing a composition of amplified complimentary DNA (cDNA) from RNA extracted amplified complimentary DNA (cDNA) from RNA extracted from a blood, plasma, serum or urine sample of a xenotransplant recipient useful for determination of xenotransplant rejection, wherein the composition comprises target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection, wherein xenotransplant rejection is determined by a combination of the amount of the one or more target loci indicating xenotransplant rejection or a function thereof, and the total amount of animal donor-derived RNA or the fraction of animal donor-derived RNA.

In some embodiments, the RNA is derived from extracellular vesicles (EVs) isolated from a blood, plasma, serum or urine samples of a xenotransplant recipient.

### Methods of Determining and Monitoring Xenotransplant Rejection Based on Measuring Protein

In one aspect the present disclosure relates to a method of preparing a composition of protein derived from a blood, plasma, serum or urine sample of a xenotransplant recipient useful for determination of xenotransplant rejection, comprising: (a) extracting protein from the blood, plasma, serum or urine sample of the xenotransplant recipient, wherein the protein comprises donor-derived protein from the xenotransplant and recipient-derived protein; (b) detecting and quantifying an amount of donor-derived protein; (c) determining whether the amount of protein donor-derived protein or a function thereof exceeds a cutoff threshold indicating xenotransplant rejection.

In one aspect the present disclosure relates to a method of preparing a composition of protein derived from a blood, plasma, serum or urine sample of a xenotransplant recipient useful for determination of xenotransplant rejection, comprising: (a) extracting protein from the blood, plasma, serum or urine sample of the xenotransplant recipient, wherein the protein comprises donor-derived protein from the xenotransplant and recipient-derived protein; (b) detecting and quantifying an amount of one or more target proteins, wherein the one or more target proteins comprise both recipient-derived proteins and donor-derived proteins, and wherein the one or more target proteins are indicative of xenotransplant rejection; (c) determining whether the amount of the one or more target proteins or a function thereof exceeds a cutoff threshold indicating xenotransplant rejection.

In one aspect the present disclosure relates to a method of preparing a composition of protein derived from extracellular vesicles (EVs) isolated from a blood, plasma, serum or urine sample of a xenotransplant recipient useful for determination of xenotransplant rejection, comprising: (a) extracting protein from extracellular vesicles (EVs) isolated from the blood, plasma, serum or urine sample of the xenotransplant recipient, wherein the protein comprises donor-derived protein from the xenotransplant and recipient-derived protein; (b) detecting and quantifying an amount of donor-derived protein; (c) determining whether the amount of protein donor-derived protein or a function thereof exceeds a cutoff threshold indicating xenotransplant rejection.

In one aspect the present disclosure relates to a method of preparing a composition of protein derived from extracellular vesicles (EVs) isolated from a blood, plasma, serum or urine sample of a xenotransplant recipient useful for determination of xenotransplant rejection, comprising: (a) extracting protein from extracellular vesicles (EVs) isolated from the blood, plasma, serum or urine sample of the xenotransplant recipient, wherein the protein comprises donor-derived protein from the xenotransplant and recipient-derived protein; (b) detecting and quantifying an amount of one or more target proteins, wherein the one or more target proteins comprise both recipient-derived proteins and donor-derived proteins, and wherein the one or more target proteins are indicative of xenotransplant rejection; (c) determining whether the amount of the one or more target proteins or a function thereof exceeds a cutoff threshold indicating xenotransplant rejection.

In one aspect the present disclosure relates to a method of administrating immunosuppressive therapy in a xenotransplant recipient, comprising: (a) measuring an amount of donor-derived protein; and (b) titrating the dosage of an immunosuppressive therapy according to the amount of donor-derived protein or a function thereof.

In one aspect the present disclosure relates to a method of administrating immunosuppressive therapy in a xenotransplant recipient, comprising: (a) measuring an amount of one or more target proteins; and (b) titrating the dosage of an immunosuppressive therapy according to the amount of the one or more target proteins or a function thereof.

In some embodiments, the method herein further comprises repeating steps (a)-(b) longitudinally for the same xenotransplant recipient, and determining a longitudinal change in the amount donor-derived protein, donor-derived target proteins, or a function thereof, and a longitudinal change in the amount of donor-derived protein, target proteins, or a function thereof.

In some embodiments, the method herein further comprises titrating the dosage of the immunosuppressive therapy according to the longitudinal change in the donor-derived protein, donor-derived target proteins, or a function thereof.

Methods of measuring protein amounts include, but are not limited to various sandwich, competitive, or non-competitive assay formats, to generate a signal that is related to the presence or amount of an protein analyte of interest. One agent for detecting a protein of the invention is e.g. an antibody capable of binding to the protein, preferably an antibody with a detectable label. Antibodies can be polyclonal, or preferably, monoclonal. An intact antibody or a fragment thereof (e.g. Fab or F(ab′)₂ can be used. The term “labeled” is intended to encompass direct labelling of the antibody by coupling a detectable substance to antibody, as well as indirect labeling of the antibody by reactivity with another reagent that is directly labeled.

A variety of formats can be employed to determine whether a sample contains a protein that binds to a given antibody. Examples of such formats include e.g. enzyme immunoassay, radioimmunoassay, Western blot analysis, and ELISA. Numerous formats for antibody arrays have been described proposed employing antibodies. Such arrays typically include different antibodies having specificity for different proteins intended to be detected. For example, usually at least one hundred different antibodies are used to detect one hundred different protein targets, each antibody being specific for one target. In some embodiments, protein amounts are measure by using mass spectrometry based approaches. In a related aspect, the invention provides arrays which contain a support or supports bearing a plurality of ligands that specifically bind to a plurality of proteins. The plurality of proteins includes at least two, three, four or five proteins determined to be indicative of transplant rejection. In some embodiments, the plurality of proteins are fewer than 1000 or fewer than 100 in number, and more than 100 or more than 10, respectively. In some embodiments, the plurality of ligands are attached to a planar support or to beads. In some embodiments, the ligands are different antibodies, and the different antibodies bind to different proteins of the plurality of proteins.

In some embodiments, the target proteins are encoded by the RNA targets disclosed elsewhere herein.

### Samples Comprising Nucleic Acids and Methods for Obtaining Samples and Extracting Nucleic Acids

The method disclosed herein comprises extracting fragmented or intact RNA derived from a sample obtained from a xenotransplant recipient. In some embodiments, the xenotransplant recipient is a human subject, and the xenotransplant donor is a pig. In some embodiments, the xenotransplant is from a pig, a primate, a baboon, a cow, or a dog.

In some embodiments, the xenotransplant recipient has received a plurality of transplanted organs selected from pancreas, kidney, liver, lung, heart, intestinal, thymus or uterus. In some embodiments, the one or more transplanted organs are from the same transplant donors. In some embodiments, the one or more transplanted organs are from more multiple different transplant donors. In some embodiments, the transplant recipient has received simultaneous transplantation of more than one organ.

In some embodiments, the xenotransplant recipient has received one or more transplanted organs selected from kidney, liver, heart, lung, pancreas, intestinal, thymus, and uterus. In some embodiments, the xenotransplant recipient has received a kidney transplant. In some embodiments, the xenotransplant recipient has received a liver transplant. In some embodiments, the xenotransplant recipient has received a heart transplant. In some embodiments, the xenotransplant recipient has received a lung transplant. In some embodiments, the xenotransplant recipient has received a pancreas transplant. In some embodiments, the xenotransplant recipient has received an intestinal transplant. In some embodiments, the xenotransplant recipient has received a thymus transplant. In some embodiments, the xenotransplant recipient has received a uterus transplant.

In some embodiments, the sample is obtained from the xenotransplant recipient less than 18 months post-transplantation, less than 17 months post-transplantation, less than 16 months post-transplantation, less than 15 months post-transplantation, less than 14 months post-transplantation, less than 13 months post-transplantation, or less than 12 months post-transplantation. In some embodiments, the sample is obtained from the transplant recipient between 0 and 2 months post-transplantation, between 2 and 4 months post-transplantation, between 4 and 6 months post-transplantation, between 6 and 9 months post-transplantation, between 9 and 12 months post-transplantation, or between 12 and 18 months post-transplantation.

In some embodiments, the methods disclosed herein further comprise measuring the amounts of cell-free DNA, RNA, or protein longitudinally for the same xenotransplant recipient; determining a longitudinal change in the amount of cell-free DNA, RNA, or protein. In some embodiments, the amounts of cell-free DNA, RNA, or protein is the total amount of cell-free DNA, RNA, or protein derived from the donor organ.

In some embodiments, the xenotransplant recipient has received one or more organs from the same transplant donors. In some embodiments, the xenotransplant recipient has received one or more organs from more multiple different transplant donors. In some embodiments, the xenotransplant recipient has received simultaneous transplantation of more than one organ.

The samples may be a body fluid sample, a tissue, an organ, or individual cells. In some embodiments the sample comprises blood, plasma, serum, CSF or urine. In some embodiments, the sample is blood. In some embodiments, the sample is blood, plasma, or scrum. In some embodiments, the samples may be extracellular vehicles derived from the body fluid samples such as blood, plasma, serum, CSF or urine.

### Nucleic Acids and Methods of Extracting or Enriching Nucleic Acids

The methods disclosed herein comprises extracting nucleic acids from a sample derived from a subject. The nucleic acids may be cell-free DNA, cellular DNA, DNA extracted from exosomes, cell-free RNA, cellular RNA, or RNA extracted from exosomes. The term “RNA” refers herein to any type of RNA, including messenger RNA (mRNA) or small non-coding RNA (sncRNA) such as micro RNA (miRNA). In some embodiments, the RNA may be cell-free, cellular, or exosome RNA. In some embodiments, the RNA comprises small non-coding RNA (sncRNA). In some embodiments, the sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the cell-free sncRNA is derived from exosomes or microvesicles.

In some embodiments, nucleic acids are extracted by using size exclusion. In some embodiments, cell-free DNA or RNA is isolated from cellular DNA or RNA based on size. In some embodiments, nucleic acids are isolated by using affinity chromatography.

In some embodiments, nucleic acids are preferentially enriched. Nucleic acids may be preferentially enriched by using preferential enrichment at a locus or target site. Such preferential enrichment refers to any method that results in the percentage of molecules of nucleic acids in a post-enrichment nucleic acid mixture that correspond to the locus being higher than the percentage of molecules of nucleic acids in the pre-enrichment nucleic acid mixture that correspond to the locus. The method may involve selective amplification of nucleic acid molecules that correspond to a locus. The method may involve removing nucleic acid molecules that do not correspond to the locus. The method may involve a combination of methods. The degree of enrichment is defined as the percentage of molecules of nucleic acids in the post-enrichment mixture that correspond to the locus or target divided by the percentage of molecules of nucleic acids in the pre-enrichment mixture that correspond to the locus or target. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

Amplification refers to a method that increases the number of copies of nucleic acid molecules. Selective Amplification may refer to a method that increases the number of copies of a particular nucleic acid molecules, or nucleic acid molecules that correspond to a particular region of nucleic acid molecules. It may also refer to a method that increases the number of copies of a particular targeted molecule of nucleic acid molecules, or targeted region of nucleic acid molecules more than it increases non-targeted molecules or regions of nucleic acid molecules.

Selective amplification may be a method of preferential enrichment. Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences. Universal Adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers. Targeting refers to a method used to selectively amplify or otherwise preferentially enrich those molecules of DNA that correspond to a set of loci, in a mixture of DNA.

Particular nucleic acids may also be enriched for by using hybrid capture. In some embodiments, preferentially enriching the RNA at the plurality of biomarkers comprises: obtaining a set of hybrid capture probes; hybridizing the hybrid capture probes to the RNA in the sample; and physically separating the hybridized RNA from the sample of RNA from the unhybridized RNA from the sample. In some embodiments, preferentially enriching the sncRNA such as miRNA at the plurality of biomarkers comprises: obtaining a set of hybrid capture probes; hybridizing the hybrid capture probes to the miRNA in the sample; and physically separating the hybridized miRNA from the sample of RNA from the unhybridized RNA from the sample. In some embodiments, preferentially enriching preselected mRNA comprises: obtaining a set of hybrid capture probes; hybridizing the hybrid capture probes to the mRNA in the sample; and physically separating the hybridized mRNA from the sample of RNA from the unhybridized RNA from the sample.

In some embodiments, in the methods disclosed herein, the DNA is preferentially enriched at the target loci or a biomarker.

The term “biomarker” refers to a molecule that is an indicator of an abnormal biological condition (e.g., disease or disorder, or transplant rejection). For example, a biomarker may be a gene product (i.e. RNA or protein) that (a) is expressed at higher or lower levels, (b) has an altered ratio relative to another biomarker, (c) is present at higher or lower levels, (d) is a variant or mutant of the gene product, or (e) is simply present or absent, in a cell or tissue sample from a subject having or suspected of having a disease as compared to an undiseased tissue or cell sample from the subject having or suspected of having a disease, or as compared to a cell or tissue sample from a subject or a pool of subjects not having or suspected of having the disease. In the context of transplant, the biomarker may be indicative of poor donor organ health or transplant rejection. That is, one or more gene products are sufficiently specific to the test sample that one or more may be used to identify, predict, or detect the presence of transplant rejection, disease, risk of disease, risk of a given event or change in disease status, or provide information for a proper or improved therapeutic regimen.

In some embodiments, one or more biomarkers can be one or a collection of genetic aberrations, which is used herein to refer to the nucleic acid amounts as well as nucleic acid variants within the nucleic acid-containing particles. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes), nucleic acid modifications (e.g., methylation, acetylation and phosphorylation), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

In some embodiments, preferentially enriching the DNA in the sample at the plurality of polymorphic loci includes obtaining a plurality of pre-circularized probes where each probe targets one of the polymorphic loci, and where the 3′and 5′end of the probes are designed to hybridize to a region of DNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to DNA from the sample, filling the gap between the hybridized probe ends using DNA polymerase, circularizing the pre-circularized probe, and amplifying the circularized probe.

In some embodiments, preferentially enriching the DNA at the plurality of polymorphic loci includes obtaining a plurality of ligation-mediated PCR probes where each PCR probe targets one of the polymorphic loci, and where the upstream and downstream PCR probes are designed to hybridize to a region of DNA, on one strand of DNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the DNA from the first sample, filling the gap between the ligation-mediated PCR probe ends using DNA polymerase, ligating the ligation-mediated PCR probes, and amplifying the ligated ligation-mediated PCR probes.

In some embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some embodiments, preferentially enriching the DNA at a plurality of polymorphic loci includes obtaining a plurality of inner forward primers where each primer targets one of the polymorphic loci, and where the 3′end of the inner forward primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′end of the inner reverse primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the DNA, and amplifying the DNA using the polymerase chain reaction to form amplicons.

In some embodiments, the method also includes obtaining a plurality of outer forward primers where each primer targets one of the polymorphic loci, and where the outer forward primers are designed to hybridize to the region of DNA upstream from the inner forward primer, optionally obtaining a plurality of outer reverse primers where each primer targets one of the polymorphic loci, and where the outer reverse primers are designed to hybridize to the region of DNA immediately downstream from the inner reverse primer, hybridizing the first primers to the DNA, and amplifying the DNA using the polymerase chain reaction.

In some embodiments, the method also includes obtaining a plurality of outer reverse primers where each primer targets one of the polymorphic loci, and where the outer reverse primers are designed to hybridize to the region of DNA immediately downstream from the inner reverse primer, optionally obtaining a plurality of outer forward primers where each primer targets one of the polymorphic loci, and where the outer forward primers are designed to hybridize to the region of DNA upstream from the inner forward primer, hybridizing the first primers to the DNA, and amplifying the DNA using the polymerase chain reaction.

In some embodiments, preparing the first sample further includes appending universal adapters to the DNA in the first sample and amplifying the DNA in the first sample using the polymerase chain reaction. In some embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some embodiments, amplifying the DNA is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some embodiments, preparing the sample further comprises dividing the sample into a plurality of portions, and where the DNA in each portion is preferentially enriched at a subset of the plurality of polymorphic loci. In some embodiments, the inner primers are selected by identifying primer pairs likely to form undesired primer duplexes and removing from the plurality of primers at least one of the pair of primers identified as being likely to form undesired primer duplexes. In some embodiments, the inner primers contain a region that is designed to hybridize either upstream or downstream of the targeted polymorphic locus, and optionally contain a universal priming sequence designed to allow PCR amplification. In some embodiments, at least some of the primers additionally contain a random region that differs for each individual primer molecule. In some embodiments, at least some of the primers additionally contain a molecular barcode.

In some embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some embodiments, the method does not comprise using a microarray.

In some embodiments, the method comprises (a) performing multiplex polymerase chain reaction (PCR) on the cell free DNA sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some embodiments, the method does not comprise using a microarray.

In some embodiments, mRNA is isolated by using probes that hybridize to the poly-A tail of the mRNA molecules.

After blood draw and before nucleic acid extraction, blood cells within a blood sample may burse and shed long fragments of DNA into the sample, which would increase the total amount of cell-free DNA (cfDNA) and background noise, distorting thd % of dd-cfDNA detected. In order to reduce such background noise, and based on the observation that dd-cfDNA is typically shorter than DNA shredded from a transplant recipient blood cell, two particular enrichments for dd-cfDNA are contemplated. In one embodiment, a size selection is applied to select for shorter cfDNA. In another embodiment, a universal amplification step is applied to reduce noise (e.g., before applying multiplex PCR), based on the hypothesis that shorter dd-cfDNA (often in mononucleosomal form) is amplified more efficiently than longer transplant recipient-derived DNA.

### Target Genes and Loci and Protein Targets

The nucleic acids may comprise biomarkers indicative of an immune response, or various diseases or conditions as described elsewhere herein. In some embodiments, the target loci comprise one or more different sets of target loci. In some embodiments, the target loci comprises a set of human target loci and a set of animal target loci, wherein the set of human target loci a is different from the set of animal target loci. In some embodiments, the set of human target loci and the set of animal target loci are the same, and one or more human target loci can be distinguished from the corresponding animal loci by an insert sequence.

In some embodiments, the method comprises extracting fragmented or intact mRNA derived from sample of the xenotransplant recipient, wherein the extracted mRNA comprises donor-and/or recipient-derived mRNA, and wherein the mRNA comprises a plurality of biomarkers indicative of an immune response, or a discase or disorder. In some embodiments, the biomarker indicates an increased immune response. In some embodiments, the biomarker indicates a decreased immune response. In some particular embodiments, sncRNA (such as miRNA) biomarkers indicate an increased immune response, or a decreased immune response.

In some embodiments, the presently disclosed method comprises pre-selecting RNA target molecules. In some embodiments, the RNA target molecules comprises RNA species known to be relevant for assessing organ health. In some embodiments, the present disclosure provides methods for identifying RNA target molecules that are relevant for assessing organ health.

In some embodiments, the methods disclosed herein further comprise preferentially enriching the RNA at a plurality of biomarkers indicative of xenotransplant rejection. In some embodiments, the RNA biomarkers indicate an increased immune response, or a decreased immune response.

In some embodiments, the biomarkers comprise single nucleotide polymorphism (SNP) loci.

### Samples and Methods for Isolating Nucleic Acids From the Samples

In some embodiments, the nucleic acid sample includes fragmented or digested nucleic acids. In some embodiments, the nucleic acid sample includes DNA, such as genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA.

In some embodiments, nucleic acid sample includes RNA, such as cfRNA, cellular RNA, cytoplasmic RNA, coding cytoplasmic RNA, non-coding cytoplasmic RNA, mRNA, miRNA, mitochondrial RNA, rRNA, or tRNA. In some embodiments, the nucleic acid sample includes DNA from a single cell, 2 cells, 3 cells, 4 cells, 5 cells, 6 cells, 7 cells, 8 cells, 9 cell, 10cells, or more than 10 cells. In some embodiments, the nucleic acid sample is a blood or plasma sample that is substantially free of cells. In some embodiments, the nucleic acid sample includes or is derived from blood, plasma, saliva, semen, sperm, cell culture supernatant, mucus secretion, dental plaque, gastrointestinal tract tissue, stool, urine, hair, bone, body fluids, tears, tissue, skin, fingernails, blastomeres, embryos, amniotic fluid, chorionic villus samples, bile, lymph, cervical mucus, or a forensic sample. In some embodiments, the target loci are segments of human nucleic acids. In some embodiments, the target loci are segments of human nucleic acids found in the human genome. In some embodiments, the target loci comprise or consist of single nucleotide polymorphisms (SNPs). In some embodiments, the primers are DNA molecules.

In some embodiments, the method includes isolating or purifying the DNA and/or RNA. There are a number of standard procedures known in the art to accomplish such an end. In some embodiments, the sample may be centrifuged to separate various layers. In some embodiments, the DNA or RNA may be isolated using filtration. In some embodiments, the preparation of the DNA or RNA may involve amplification, separation, purification by chromatography, liquid separation, isolation, preferential enrichment, preferential amplification, targeted amplification, or any of a number of other techniques either known in the art or described herein. In some embodiments for the isolation of DNA, RNase is used to degrade RNA. In some embodiments for the isolation of RNA, DNase (such as DNase I from Invitrogen, Carlsbad, Calif., USA) is used to degrade DNA. In some embodiments, an RNEASY™ mini kit (Qiagen), is used to isolate RNA according to the manufacturer's protocol. In some embodiments, small RNA molecules are isolated using the MIRVANA™ PARIS kit (Ambion, Austin, Tex., USA) according to the manufacturer's protocol (Gu et al., J. Neurochem. 122:641-649, 2012, which is hereby incorporated by reference in its entirety). The concentration and purity of RNA may optionally be determined using Nanovue (GE Healthcare, Piscataway, N.J., USA), and RNA integrity may optionally be measured by use of the 2100 Bioanalyzer (Agilent Technologies, Santa Clara, Calif., USA) (Gu et al., J. Neurochem. 122:641-649, 2012, which is hereby incorporated by reference in its entirety). In some embodiments, TRIZOL or RNALATER™ (Ambion) is used to stabilize RNA during storage.

In some embodiments, adaptors are added to make a sequencing library. Prior to ligation, sample DNA may be blunt ended, and then a single adenosine base is added to the 3-prime end. In some embodiments, ligation of adaptors to nucleic acids is a sticky end ligation. Prior to ligation the DNA may be cleaved using a restriction enzyme or some other cleavage method. During ligation the 3-prime adenosine of the sample fragments and the complementary 3-prime tyrosine overhang of adaptor can enhance ligation efficiency. In some embodiments, adaptor ligation is performed using the ligation kit found in the AGILENT SURESELECT™ kit.

In some embodiments, the library is amplified using universal primers. In an embodiment, the amplified library is fractionated by size separation or by using products such as AGENCOURT AMPURE™ beads or other similar methods. In some embodiments, PCR amplification is used to amplify target loci. In some embodiments, the amplified DNA is sequenced (such as sequencing using an ILLUMINA IIGAX™ or HiSeq sequencer). In some embodiments, the amplified DNA is sequenced from each end of the amplified DNA to reduce sequencing errors. If there is a sequence error in a particular base when sequencing from one end of the amplified DNA, there is less likely to be a sequence error in the complementary base when sequencing from the other side of the amplified DNA (compared to sequencing multiple times from the same end of the amplified DNA).

In some embodiments, miRNA can be separated from fragments of RNA caused by degradation because degraded RNA has lost phosphorylation groups at the ends. The miRNA retains the phosphorylation groups at the ends. An adapter can be ligated to phosphorylated miRNA ends, but the adaptor will not ligate to unphosphorylated RNA species such as degraded mRNA. The adaptor can contain sequences that allow for primer binding to aid reverse transcription to produce complementary DNA (cDNA) selectively from.

As nonlimiting examples, a locus can be a single nucleotide polymorphism, an intron, or an exon. In some embodiments, a locus can include an insertion, deletion, or transposition. In some embodiments, the sample can include a blood, sera, or plasma sample. In some embodiments, the sample can include free floating DNA (e.g. circulating cell-free tumor DNA or circulating cell-free fetal DNA) in a blood, sera, or plasma sample. In these embodiments, the sample is typically from an animal, such as a mammal or human, and is typically present in fragments about 160 nucleotides in length. In some embodiments, the free-floating DNA is isolated from blood using an EDTA-2Na tube after removal of cellular debris and platelets by centrifugation. The plasma samples can be stored at −80.degree. C. until the DNA is extracted using, for example, QIAAMP™ DNA Mini Kit (Qiagen, Hilden, Germany), (e.g. Hamakawa et al., Br J Cancer. 2015; 112:352-356). However, the sample can be derived from other sources and nucleic acid molecules from any organism can be used for this method. In some embodiments, DNA derived from bacteria and/or viruses can be used to analyze true sequence variants within a mixed population, especially in environmental and biodiversity sampling.

Many kits and methods are known in the art for generating libraries of nucleic acid molecules for subsequent sequencing. Kits especially adapted for preparing libraries from small nucleic acid fragments, especially circulating cell-free DNA, can be useful for practicing methods provided herein. For example, the NEXTFLEX™M Cell Free kits (Bioo Scientific, Austin, Tex.) or the Natera Library Prep Kit (Natera, San Carlos, Calif.). Such kits would typically be modified to include adaptors that are customized for the amplification and sequencing steps of the methods provided herein. Adaptor ligation can also be performed using commercially available kits such as the ligation kit found in the Agilent SURESELECT™ kit (Agilent, Santa Clara, Calif.).

Sample nucleic acid molecules are composed of naturally occurring or non-naturally occurring ribonucleotides or deoxyribonucleotides linked through phosphodiester linkages. Furthermore, sample nucleic acid molecules are composed of a nucleic acid segment that is targeted for sequencing. Sample nucleic acid molecules can be or can include nucleic acid segments that are at least 20, 25, 50, 75, 100, 125, 150, 200, 250, 300, 400, 500, 600, 700, 800, 900, or 1,000 nucleotides in length. In any of the embodiments disclosed herein the sample nucleic acid molecules or nucleic acid segments can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the nucleic acid molecules can be fragments of genomic DNA and can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. For the sake of clarity, nucleic acids initially isolated from a living tissue, fluid, or cultured cells, can be much longer than sample nucleic acid molecules processed using methods herein. As discussed herein, for example, such initially isolated nucleic acid molecules can be fragmented to generate nucleic acid segments, before being used in the methods herein. In some embodiments, the nucleic acid molecules and nucleic acid segments can be identical. The sample nucleic acid molecule or sample nucleic acid segment can include a target locus that contains the nucleotide or nucleotides that are being queried, especially a single nucleotide polymorphism or single nucleotide variant. In any of the disclosed embodiments, the target loci can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the sample nucleic acid molecule and/or the sample nucleic acid segment. In other embodiments, the target loci can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the target loci on different sample nucleic acid molecules can be at least 50%, 60%, 70%, 80%, 90% 95%, 96%, 97%, 98%, 99%, 99.9%, or 100% identical. In some embodiments, the target loci on different sample nucleic acid molecules can share at least 50%, 60%, 70%, 80%, 90% 95%, 96%, 97%, 98%, 99%, 99.9%, or 100% sequence identity.

In some embodiments, the entire sample nucleic acid molecule is a sample nucleic acid segment. For example, in certain embodiments where adaptors are ligated directly to the ends of sample nucleic acid molecules, or ligated to a nucleic acid(s) ligated to the ends of sample nucleic acid molecules, or ligated as part of primers that bind to sequences at the termini of sample nucleic acid segments, or adapters, such as universal adapters added thereto, as discussed further herein, the entire nucleic acid molecule can be a sample nucleic acid segment. In other embodiments, for example certain embodiments where adaptors are attached to sample nucleic acid molecules as part of primers that target binding sites internal to the termini of sample nucleic acid molecules, a portion of the sample nucleic acid molecule can be the sample nucleic acid segment that is targeted for downstream sequencing. For example, at least 50%, 60%, 70%, 75%, 80%, 85%, 90%, 95%, 96%, 97%, 98%, 99%, or 100% of a sample nucleic acid molecule can be a nucleic acid segment.

In some embodiments, sample nucleic acid molecules are a mixture of nucleic acids isolated from a natural source, some sample nucleic acid molecules having identical sequences, some having sequences sharing at least 50%, 60%, 70%, 80%, 90%, 95%, 98%, or 99% sequence identity, and some with less than 50%, 40%, 30%, 20%, 10%, or 5% sequence identity over between 20, 25, 50, 75, 100, 125, 150, 200, 250 nucleotides on the low end of the range, and 50,75, 100, 125, 150, 200, 250, 300, 400, or 500 nucleotides on the high end of the range. Such sample nucleic acid molecules can be nucleic acid samples isolated from tissues or fluids of a mammal, such as a human, without enriching one sequence over another. In other embodiments, target sequences, for example, those from a gene of interest, can be enriched prior to performing methods provided herein.

### Removing Contamination

Biological samples may contain large amounts of contaminating nucleic acids that can make detection of target nucleic acids difficult, reduce quality of the nucleic acid library, and/or reduce the throughput of the assay. For example, blood cells within a blood sample may burst and shed nucleic acids into the sample, resulting in that the sample may comprise large amount of irrelevant nucleic acids form the red blood cells. In one aspect of the present disclosure, the genome editing CRISPR-Cas system may be used to remove contaminating nucleic acids. CRISPR-Cas technology allows for simple, flexible, and relatively inexpensive cutting of DNA and/or RNA in a targeted sequence-specific manner. The required components for in vitro digestion comprises a Cas effector protein and a target-specific guide RNA (gRNA)).

An effective CRISPR-Cas complex must have several features. First, the gRNA must have a spacer element which is complementary to the target nucleic acids. This complementary target nucleic acid sequence is referred to as the protospacer. In addition, immediately downstream of the spacer (For Cas9, for Cas12a it is immediately upstream of the spacer), there must be a Protospacer Adjacent Motif (PAM). For Cas9 this motif can be NGG, where N is any nucleotide, while for Cas12a the PAM motif can be TTTV, where V is any nucleotide except T. Also required for proper complex formation is the scaffold of the gRNA. However, since this is conserved for all gRNAs for the same Cas enzyme, IDT has standardized this for Cas9 and created a separate “tracrRNA” which anneals to the shorter “crRNA” to form the complete gRNA.

Various CRISPR Cas systems have been developed. The CRISPR Cas9 system is the first and best-characterized single-protein CRISPR effector and are subcategorized in to types II-A, II-B, and II-C. Cas9 makes a blunt double-stranded DNA break, which can then be repaired by cither non-homologous end joining or homologous recombination with a donor template DNA to create site-specific edits. Type II-A Cas9s generally have high genome editing efficiency, but off-target cleavage at unintended genome sites can be a disadvantage. Variants have been engineered to overcome these limitations, and type II-C Cas9s tend to have naturally higher fidelity. Cas9 uses a guide spacer with a length of 18-24 nucleotides (nt). The total length of the guide for Cas9 may be about 100 nt. The PAM sequence may be 3-NGG (SpCas9), 3-NNGRRT (SaCas9), 3-NNNNGATT (NmCas9). Cas9 produces a blunt-ended dsDNA break. Mutant Cas9enzymes are also commercially available which produce single-stranded nicks in DNA on one strand of the target, as well as a non-cutting mutant which only binds the target.

Cas12 belongs to Type V CRISPR-Cas, which comprises subtypes V-A and V-B, also known as Cpfl (Type V-A), or C2c1 (type V-B), among other subtypes. Cas12 is a compact and efficient enzyme that creates staggered cuts in dsDNA, and thereby creates a 3 to 5 base overhang. Guide spacer of Cas12 has a length of 18-25 nt, and the total guide length is 42-44 nt. The Cas12 PAM sequences may be 5-TTTN (FnCas12a). Cas12 processes its own guide RNAs, leading to increased multiplexing ability. Cas12 has also been engineered as a platform for epigenome editing, and it was recently discovered that Cas12a can indiscriminately chop up single-stranded DNA once activated by a target DNA molecule matching its spacer sequence.

Cas13 is a Type VI CRISPR-Cas that comprises subtypes VI-A, VI-B, VI-C, and VI-D, also known as C2c2(VI-A) or CasRx (type VI-D). Cas13 targets RNA, not DNA. The guide spacer for Cas13 has a length of 22-30 nt, and a total guide length of 52-66 nt. The Cas13 PAM sequences included 3-H (LshCas13a), 5-D, and 3-NAN or NNA (BzCas13b), and none (RfCas13d). Once Cas13 is activated by a ssRNA sequence bearing complementarity to its crRNA spacer, Cas13 exhibits a nonspecific RNase activity to destroy all nearby RNA regardless of their sequence. This property has been harnessed in vitro for precision diagnostics. These systems can also be used for efficient, multiplexable, and specific RNA knockdown or RNA sequence editing in mammalian cells.

The methods disclosed herein may comprise utilizing any CRISPR-Cas system to deplete contaminating or overabundant nucleic acids in a biological sample or libraries. For example Cas9 and/or Cas12 may be used to remove DNA species in whole blood or hemolysis tainted blood, scrum or plasma samples, thereby increasing the sensitivity of detecting the target loci. In another aspect, the Cas enzymes targeting RNA species directly may be used. For example, Cas13 may be used to remove contaminating RNA species from the biological sample prior to library preparation.

Cas enzymes can be given a mixture of different gRNAs to allow targeting of many different loci in the same reaction. In some embodiments, 1 gRNA is used to target a loci. In some embodiments, 1-5 gRNAs are used to target 1-5 loci in the same reaction. In some embodiments, 1-10 gRNAs are used to target 1-10 loci in the same reaction. In some embodiments, 1-100 gRNAs are used to target 1-100 loci in the same reaction. In some embodiments, 1-5000 gRNAs are used to target 1-5000 loci in the same reaction. In some embodiments, 10-5000 gRNAs are used to target 10-5000 loci in the same reaction. In some embodiments, 100-5000 gRNAs are used to target 100-5000 loci in the same reaction. In some embodiments, 1000-50000 gRNAs are used to target 1000-50000 loci in the same reaction. In some embodiments, 10000-5000 gRNAs are used to target 10000-50000 loci in the same reaction. In some embodiments, a plurality of gRNAs are used to target a plurality of target loci in the same reaction. In some embodiments, at least 10gRNAs are used to target at least 10 loci in the same reaction. In some embodiments, at least 20 gRNAs are used to target at least 20 loci in the same reaction. In some embodiments, at least 50 gRNAs are used to target at least 50 loci in the same reaction. In some embodiments, at least 100gRNAs are used to target at least 100 loci in the same reaction. In some embodiments, at least 500 gRNAs are used to target at least 500 loci in the same reaction. In some embodiments, at least 1000 gRNAs are used to target at least 1000 loci in the same reaction. In some embodiments, at least 2000 gRNAs are used to target at least 2000 loci in the same reaction. In some embodiments, at least 3000 gRNAs are used to target at least 3000 loci in the same reaction. In some embodiments, at least 4000 gRNAs are used to target at least 4000 loci in the same reaction. In some embodiments, at least 5000 gRNAs are used to target at least 5000 loci in the same reaction. In some embodiments, at least 10000 gRNAs are used to target at least 10000 loci in the same reaction. In some embodiments, at least 20000 gRNAs are used to target at least 20000 loci in the same reaction.

Untargeted miRNA analysis may be performed by ligating the miRNA to an adaptor as outlined. The adaptor may provide a NGG location adjacent to the miRNA to allow for Cas9 cutting. When an unwanted sequence is ligated to the adaptor, a Cas9-gRNA complex complementary to this unwanted sequence will be introduced. This will result in removal of the adaptor, and preventing PCR amplification. This step is performed on double-stranded reverse transcribed DNA.

In some embodiments, a pilot sequencing run can be used to inform which targets need to be removed from future sequencing runs. This would allow assay developers to focus on only miRNAs of interest by designing gRNAs targeting the most common uninteresting or contaminating small RNA fragments actually observed in the specific sample type used in the assay. This could include highly abundant miRNAs or small fragments of ribosomal or messenger RNAs that might cause background noise.

In some embodiments, Cas enzymes can be removed by using thermolabile Proteinase K (NEB P8111S) because Cas complexes are relatively long-lived and may interfere with downstream applications.

While this approach could also be used in targeted miRNA applications, it is preferable to improve/remove bad primers prior to the assay rather than try to remove them after amplification using CRISPR-Cas.

In some embodiments, the CRISPER-Cas mediated removal of contaminating species may also be used in mRNA applications. In some embodiments, CRISPR-Cas approaches are used to remove contaminating mRNA species with a length less than 20 nucleotides. When the length of the contaminating mRNA species is less than 20 nucleotides, a similar approach to that proposed for miRNA could be used to design assays to specifically target the observed sequences. This could be especially useful if, for example, barcoding primer dimers are consuming a large portion of the sequencing reads.

In some embodiments, CRISPR-Cas approaches are used to remove contaminating mRNA species with a length larger than 20 nucleotides. In some embodiments, CRISPR-Cas approaches are used in combination with a “tag and capture” method to remove contaminating mRNA species with a length larger than 20 nucleotides.

In some embodiments, the CRISPR-Cas method to remove contaminating species is used on cDNA derived from miRNA or mRNA prior to amplification. In some embodiments, the CRISPR-Cas method to remove contaminating species is used on cDNA derived from miRNA or mRNA, and wherein the cDNA is amplified for 1 to 5 cycles, 1 to 10 cycles, or 1-15, cycles. In some embodiments, the cDNA is amplified for no more than 2, 3, 4, 5, 6, 7, 8, 9, or 10 cycles prior to CRISPR-Cas mediated removal of contaminating species.

In some embodiments, the contaminating or overabundant species comprise hemoglobin mRNA, tRNA, rRNA, and miRNAs such as miR-451, miR-144, and miR-486, thereby increasing the fraction of desired reads that map to a target loci of interest per sample and the sample throughput per sequencing run.

In some embodiments, the method further comprises depleting adaptor dimers, primer dimers, unwanted ligation products from the composition of amplified nucleic acids comprising target loci, thereby increasing the fraction of desired reads that map to a target loci of interest per sample and the sample throughput per sequencing run.

### Methods of Identification of Biomarkers of Organ Health Transplant Interest

Text mine databases to identify biomarkers of known interest to transplant health. Identify biomarkers related to transplant organ health and look for a common signature versus randomly selected biomarker sets. Artificial intelligence may be used for text mining and predicting biomarkers of known interest for transplant rejection and organ health.

### Combining Measuring Cell-Free DNA With Measuring RNA to Determine and/or Monitoring Transplant Rejection

In some embodiments, the method herein further comprises: (i) measuring the amount of donor-derived cell-free DNA in a sample obtained from the xenotransplant recipient, extracting cell-free DNA from the sample obtained from the xenotransplant recipient, wherein the extracted cell-free DNA comprises donor-derived cell-free DNA and recipient-derived cell-free DNA; (ii) performing targeted amplification of the extracted DNA at 50-50,000 target loci in a single reaction volume; (iii) sequencing the amplified DNA by high-throughput sequencing to obtain sequencing reads and quantifying the amount of donor-derived cell-free DNA based on the sequencing reads, determining xenotransplant rejection based on whether the amount of donor-derived cell-free DNA or a function thereof exceeds a cutoff threshold of cell-free DNA amount that indicates xenotransplant rejection, wherein xenotransplant rejection is determined based on whether both the amount of donor-derived RNA and the amount of donor derived cell-free DNA or function thereof exceeds a cutoff threshold that indicates xenotransplant rejection. The combination of the amount of RNA and cfDNA in the samples obtained from the xenotransplant recipient can serve as a biomarker for rejection and as a biomarker for net state of immunosuppression. In some embodiment, the combination of an amount of mRNA targets selected from a group of preselected targets in combination with an amount of cfDNA in the samples indicates transplant rejection. In another aspect, a rejection risk for the transplant recipient can be determined based on the amount of donor-derived RNA and/or amount of cell-free DNA. In another aspect, a rejection risk for the transplant recipient can be determined based on the amount of donor-derived mRNA and/or amount of cell-free DNA. In another aspect, a rejection risk for the transplant recipient can be determined based on the amount of donor-derived miRNA and/or amount of cell-free DNA.

### Determination of Rejection Risk for the Transplant Recipient

In some embodiments, wherein the rejection risk for the xenotransplant recipient is determined using logistic regression, random forest, or decision tree machine learning analysis. In some embodiments, the machine learning analysis incorporates the amount of donor-derived RNA in the sample of the transplant recipient or a function thereof as a parameter. In some embodiments, the machine learning analysis incorporates the number of reads of donor-derived RNA or a function thereof as a parameter. In some embodiments, the machine learning analysis incorporates the estimated percentage of donor-derived RNA out of total RNA as a parameter. In some embodiments, the machine learning analysis incorporates the amount of cell-free DNA, the number of reads of cell-free DNA, or the estimated percentage of cell-free DNA out of total cell-free DNA in the sample of the transplant recipient as a parameter. In some embodiments, the machine learning analysis incorporates the amount of total amount of a plurality of proteins derived from the xenotransplant. In some embodiments, the machine learning analysis further incorporates the amount of total cell-free DNA in the sample of the transplant recipient or a function thereof as a parameter. In some embodiments, the machine learning analysis further incorporates the number of reads of total cell-free DNA or a function thereof as a parameter.

Machine learning may be used to resolve rejection vs non-rejection. Machine learning is disclosed in WO2020/018522, titled “Methods and Systems for calling Ploidy States using a Neural Network” and filed on Jul. 16, 2019 as PCT/US2019/041981, which is incorporated herein by reference in its entirety. In some embodiments, the cutoff threshold value is scaled according to the amount of total cfDNA or RNA in the blood sample.

In some embodiments, the cutoff threshold value is expressed as percentage of dd-cfDNA (dd-cfDNA %) in the blood sample. In some embodiments, the cutoff threshold value is expressed as quantity or absolute quantity of dd-cfDNA. In some embodiments, the cutoff threshold value is expressed as quantity or absolute quantity of dd-cfDNA per volume unit of the blood sample. In some embodiments, the cutoff threshold value is expressed as quantity or absolute quantity of dd-cfDNA per volume unit of the blood sample multiplied by body mass, BMI, or blood volume of the transplant recipient.

In some embodiments, the cutoff threshold value takes into account the body mass, BMI, or blood volume of the patient. In some embodiments, the cutoff threshold value takes into account one or more of the following: donor genome copies per volume of plasma, cell-free DNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some embodiments, the method has a sensitivity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled or adjusted according to the amount of total cfDNA in the blood sample and a confidence interval of 95%.

In some embodiments, the method has a specificity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled or adjusted according to the amount of total cfDNA in the blood sample and a confidence interval of 95%.

Some embodiments of the present invention relate to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

Some embodiments use either a fixed threshold of donor DNA per plasma volume or one that is not fixed, such as adjusted or scaled as noted herein. The way that this is determined can be based on using a training data set to build an algorithm to maximize performance. It may also take into account other data such as patient weight, age, or other clinical factors.

In some embodiments, the method further comprises determining the occurrence or likely occurrence of transplant rejection using the amount of donor-derived cell-free DNA. In some embodiments, the amount of donor-derived cell-free DNA is compared to a cutoff threshold value to determine the occurrence or likely occurrence of transplant rejection, wherein the cutoff threshold value is adjusted or scaled according to the amount of total cell-free DNA. In some embodiments, the cutoff threshold value is a function of the number of reads of the donor-derived cell-free DNA.

In some embodiments, the method comprises applying a scaled or dynamic threshold metric that takes into account the amount of total cfDNA in the samples to more accurately assess transplant rejection. In some embodiments, the method further comprises flagging the sample if the amount of total cell-free DNA is above a pre-determined value. In some embodiments, the method further comprises flagging the sample if the amount of total cell-free DNA is below a pre-determined value.

The RNA, DNA, or protein may be extracted from a sample from the transplant recipient, wherein the sample comprises blood, plasma, serum, Cerebrospinal fluid (CSF), or urine.

In some embodiments, the machine learning analysis further incorporates time post-transplantation as a parameter. In some embodiments, the machine learning analysis further incorporates the age of transplant recipient and/or transplant donor as a parameter. In some embodiments, the machine learning analysis further incorporates the gender of transplant recipient and/or transplant donor as a parameter.

In some embodiments, the rejection risk for the transplant recipient is determined with a sensitivity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with a specificity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with an area under the curve (AUC) of at least at least 0.86, or at least 0.87, or at least 0.88, or at least 0.89, or at least 0.90, or at least 0.91 or at least 0.92, or at least 0.93, or at least 0.94, or at least 0.95.

### Methods for Measuring the Amount of Nucleic Acids

In some embodiments, the amount of RNA is measured by quantitative PCR. In some embodiments, the amount of RNA is measured by real-time PCR. In some embodiments, the amount of RNA is measured by digital PCR. In some embodiments, the amount of RNA is measured by sequencing such as high-throughput sequencing, next-generation sequence, or sequencing-by-synthesis.

In some embodiments, the amount of donor-derived nucleic acids (e.g. RNA and/or DNA) is determined by using ratiometric and/or machine learning-artificial intelligence comparisons at a single or a plurality of time points. In some embodiments, the amount of donor-derived mRNA is determined by using ratiometric and/or machine learning-artificial intelligence comparisons at a single or a plurality of time points. In some embodiments, the amount of donor-derived miRNA is determined by using ratiometric and/or machine learning-artificial intelligence comparisons at a single or a plurality of time points.

In some embodiments, the amount of RNA or cell-free DNA is measured by a quantitative PCR method. In some embodiments, the amount of mRNA is measured by a quantitative PCR method. In some embodiments, the amount of miRNA is measured by a quantitative PCR method. In some embodiments, the quantitative PCR method comprises real-time PCR or digital PCR.

In some embodiments, the amount of mRNA or cell-free DNA is measured by massively multiplex PCR (mmPCR) to obtain amplicons comprising biomarkers, and sequencing of the amplicons.

In some embodiments, the amount of nucleic acids (e.g. mRNA, miRNA, or cell-free DNA) is measured by using microarray.

In some embodiments, the amount of nucleic acids (e.g. mRNA, miRNA, or cell-free DNA) is measured by using molecular barcodes and microscopic imaging (such as NanoString NCOUNTER®).

In some embodiments, the amount of donor-derived RNA is measured by: extracting RNA from the blood, plasma, serum, cerebral spinal fluid (CSF), or urine sample of the transplant recipient, wherein the extracted RNA comprises donor-derived RNA and recipient-derived RNA; performing targeted amplification of the extracted RNA at 100-50,000 target loci in a single reaction volume; sequencing the amplified RNA by high-throughput sequencing to obtain sequencing reads and quantifying the amount of donor-derived RNA based on the sequencing reads. In some embodiments, the amplification of RNA comprises performing reverse transcriptase to obtain complementary DNA (cDNA).

In some embodiments, the amount of donor-derived cell-free DNA is measured by: extracting cell-free DNA from the blood, plasma, serum cerebral spinal fluid (CSF), or urine sample of the transplant recipient, wherein the extracted cell-free DNA comprises donor-derived cell-free DNA and recipient-derived cell-free DNA; performing targeted amplification of the extracted DNA at 10-50,000 target loci in a single reaction volume; sequencing the amplified DNA by high-throughput sequencing to obtain sequencing reads and quantifying the amount of donor-derived cell-free DNA based on the sequencing reads.

In some embodiments, the method is performed without prior knowledge of donor genotypes. In some embodiments, the method does not comprise genotyping transplant donor(s).

In some embodiments, the amount of nucleic acids is measured by targeted amplification. In some embodiments, the amount of a particular mRNA target is measured by targeted amplification. In some embodiments, the targeted amplification comprises PCR. In some embodiments, the primers for the targeted amplification include 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 pairs of forward and reverse PCR primers. In some embodiments, the targeted amplification comprises performing amplification at 100-20,000, 500-20,000, 1,000-10,000, 200-500, 500-1,000, 1,000-2,000, 2,000-5,000, 5,000-10,000, 10,000-20,000, 20,000-50,000 target loci in a single reaction volume using 500-20,000, 1,000-10,000, 200-500, 500-1,000, 1,000-2,000, 2,000-5,000, 5,000-10,000, 10,000-20,000, or 20,000-50,000 primer pairs to obtain amplification products.

In some embodiments, the targeted amplification comprises nested PCR. In some embodiments, the primers for the targeted amplification include a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers, and a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers to obtain amplification products. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers to obtain amplification products. In some embodiments, the methods disclosed herein comprise PCR amplification of at least 10, at least 100, at least 500, at least 1000, at least 2000 biomarkers, from 10-1000, 100-10000, 200-50000, or 500-20000 RNA biomarkers, using at least 10, at least 100, at least 500, at least 1000, at least 2000, from 10-1000, 100-10000, 200-50000, 500-20000 pairs of forward and reverse PCR primers. In some embodiments, step (b) comprises amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some embodiments, the method further comprises attaching tags to the amplification products prior to performing high-throughput sequencing, wherein the tags comprise sequencing-compatible adaptors. In some embodiments, the method further comprises attaching tags to the extracted RNA prior to performing targeted amplification, wherein the tags comprise adaptors for amplification. In some embodiment, the tags comprise sample-specific barcodes, and wherein the method further comprises pooling the amplification products from a plurality of samples prior to high-throughput sequencing and sequencing the pool of amplification products together in a single run during the high-throughput sequencing.

In some embodiments, the amount of nucleic acids is determined by using for example, tracer nucleic acids, or internal calibration nucleic acids. The terms “tracer nucleic acids,” or “internal calibration nucleic acids” are used interchangeably and refer to a composition of nucleic acids for which one or more of the following is known advance-length, sequence, nucleotide composition, quantity, or biological origin. The tracer can be added to a biological sample derived from a human subject to help estimate the amount of total RNA or cfDNA in said sample. It can also be added to reaction mixtures other than the biological sample itself.

### Cutoff Threshold for Determining Transplant Rejection

In some embodiments, the cutoff threshold is an estimate percentage of donor-derived RNA out of total RNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA (e.g. mRNA or miRNA). In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA. In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived RNA out of total RNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived mRNA out of total mRNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and mRNA In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived preselected mRNA targets out of total mRNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and the preselected mRNA targets. In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived protein out of total protein or a function thereof. In some embodiments, the cutoff threshold is 1.0%, 1.1%, 1.2%, 1.3%, 1.4%, 1.5%, 1.6%, 1.7%, 1.8%, 1.9%, or 2.0% protein. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and protein. In some embodiments, the cutoff threshold is adjusted according to the type of organs transplanted. In some embodiments, the cutoff threshold is adjusted according to the number of organs transplanted.

In some embodiments, the cutoff threshold is proportional to an absolute donor-derived RNA concentration. In some embodiments, the cutoff threshold is a copy number of donor-derived RNA or a function thereof. In some embodiments, the cutoff threshold is expressed as quantity or absolute quantity of RNA. In some embodiments, the cutoff threshold is expressed as quantity or absolute quantity of RNA per volume unit of the blood sample. In some embodiments, the cutoff threshold is expressed as quantity or absolute quantity of RNA per volume unit of the blood sample multiplied by body mass, BMI, or blood volume of the transplant recipient.

In some embodiments, the cutoff threshold is proportional to an absolute donor-derived RNA concentration. In some embodiments, the cutoff threshold is a copy number of donor-derived RNA or a function thereof. In some embodiments, the cutoff threshold is expressed as quantity or absolute quantity of RNA. In some embodiments, the cutoff threshold is expressed as quantity or absolute quantity of RNA per volume unit of the blood sample. In some embodiments, the cutoff threshold is expressed as quantity or absolute quantity of RNA per volume unit of the blood sample multiplied by body mass, BMI, or blood volume of the transplant recipient.

In some embodiments, the cutoff threshold is proportional to an absolute donor-derived protein concentration. In some embodiments, the cutoff threshold is expressed as quantity or absolute quantity of protein. In some embodiments, the cutoff threshold is expressed as quantity or absolute quantity of protein per volume unit of the blood sample. In some embodiments, the cutoff threshold is expressed as quantity or absolute quantity of protein per volume unit of the blood sample multiplied by body mass, BMI, or blood volume of the transplant recipient.

In some embodiments, the method further comprises repeating steps (a)-(d) longitudinally for the same transplant recipient, and determining a longitudinal change in the amount of RNA or a function thereof and a longitudinal change in the amount of donor-derived RNA or a function thereof. In some embodiments, the method further comprises repeating steps (a)-(d) longitudinally for the same transplant recipient, and determining a longitudinal change in the amount of protein or a function thereof and a longitudinal change in the amount of donor-derived protein or a function thereof.

### Definitions

As used herein the term “single nucleotide polymorphism (SNP)” refers to a single nucleotide that may differ between the genomes of two members of the same species. The usage of the term does not imply any limit on the frequency with which each variant occurs.

In some embodiments, for example, sequence refers to a DNA or RNA sequence or a genetic sequence. It may refer to the primary, physical structure of the DNA or RNA molecule or strand in an individual. It may refer to the sequence of nucleotides found in that DNA or RNA molecule, or the complementary strand to the DNA or RNA molecule. It may refer to the information contained in the DNA or RNA molecule as its representation in silico.

In some embodiments, for example, locus refers to a particular region of interest on the DNA or RNA of an individual and includes without limitation one or more SNPs, the site of a possible insertion or deletion, or the site of some other relevant genetic variation. Disease-linked SNPs may also refer to disease-linked loci.

In some embodiments, for example, polymorphic allele, also “polymorphic locus,” refers to an allele or locus where the genotype varies between individuals within a given species. Some examples of polymorphic alleles include single nucleotide polymorphisms (SNPs), short tandem repeats, deletions, duplications, and inversions.

In some embodiments, for example, allele refers to the nucleotides or nucleotide sequence occupying a particular locus.

In some embodiments, for example, genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

In some embodiments, for example, genetic material also “genetic sample” refers to physical matter, such as tissue or blood, from one or more individuals comprising nucleic acids (e.g., comprising DNA or RNA)

In some embodiments, for example, allelic data refers to a set of genotypic data concerning a set of one or more alleles. It may refer to the phased, haplotypic data. It may refer to SNP identities, and it may refer to the sequence data of the nucleic acid, including insertions, deletions, repeats and mutations.

In some embodiments, for example, allelic state refers to the actual state of the genes in a set of one or more alleles. It may refer to the actual state of the genes described by the allelic data.

In some embodiments, for example, allelic ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.

In some embodiments, for example, allele count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

In some embodiments, for example, primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow amplification such as PCR amplification. A primer may also contain a molecular barcode. A primer may contain a random region that differs for each individual molecule.

In some embodiments, for example, hybrid capture probe refers to any nucleic acid sequence, possibly modified, that is generated by various methods such as PCR or direct synthesis and intended to be complementary to one strand of a specific target DNA or RNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.

In some embodiments, for example, sequence read refers to data representing a sequence of nucleotide bases that were measured using a clonal sequencing method. Clonal sequencing may produce sequence data representing single, or clones, or clusters of one original DNA or RNA molecule. A sequence read may also have associated quality score at each base position of the sequence indicating the probability that nucleotide has been called correctly.

In some embodiments, for example, mapping a sequence read is the process of determining a sequence read's location of origin in the genome sequence of a particular organism. The location of origin of sequence reads is based on similarity of nucleotide sequence of the read and the genome sequence.

In some embodiments, for example, DNA or RNA of donor origin refers to DNA or RNA that was originally part of a cell whose genotype was essentially equivalent to that of the transplant donor. The donor can be a human or a non-human mammalian (e.g., pig).

In some embodiments, for example, DNA or RNA of recipient origin refers to DNA or RNA that was originally part of a cell whose genotype was essentially equivalent to that of the transplant recipient.

In some embodiments, RNA may refer to messenger RNA (mRNA), small non-coding RNA (sncRNA), transfer RNA (tRNA), or a non-protein coding RNA from cells. In some embodiments, sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the RNA is cell-free RNA. In some embodiments, the cell-free RNA is derived from exosomes or microvesicles.

In some embodiments, amplification of RNA comprises reverse transcription of RNA to produce complementary DNA (cDNA) followed by amplification of cDNA by amplification methods disclosed elsewhere herein.

In some embodiments, for example, transplant recipient plasma refers to the plasma portion of the blood from a female from a patient who has received an allograft or xenograft, e.g., an organ transplant recipient.

In some embodiments, for example, preferential enrichment of DNA or RNA that corresponds to a locus, or preferential enrichment of DNA or RNA at a locus, refers to any technique that results in the percentage of molecules of DNA or RNA in a post-enrichment DNA or RNA mixture that correspond to the locus being higher than the percentage of molecules of DNA or RNA in the pre-enrichment DNA or RNA mixture that correspond to the locus. The technique may involve selective amplification of DNA or RNA molecules that correspond to a locus. The technique may involve removing DNA or RNA molecules that do not correspond to the locus. The technique may involve a combination of methods. The degree of enrichment is defined as the percentage of molecules of DNA or RNA in the post-enrichment mixture that correspond to the locus divided by the percentage of molecules of DNA or RNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

In some embodiments, for example, amplification refers to a technique that increases the number of copies of a molecule of RNA and/or DNA.

In some embodiments, for example, selective amplification may refer to a technique that increases the number of copies of a particular molecule of RNA and/or DNA, or molecules of RNA and/or DNA that correspond to a particular region of RNA and/or DNA. It may also refer to a technique that increases the number of copies of a particular targeted molecule of RNA and/or DNA, or targeted region of RNA and/or DNA more than it increases non-targeted molecules or regions of RNA and/or DNA. Selective amplification may be a method of preferential enrichment.

In some embodiments, for example, universal priming sequence refers to a DNA sequence that may be appended to a population of target nucleic acid molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.

In some embodiments, for example, universal adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

In some embodiments, for example, targeting refers to a method used to selectively amplify or otherwise preferentially enrich those molecules of DNA or RNA that correspond to a set of loci in a mixture of DNA or RNA.

### Analysis of Donor-Derived RNA for Monitoring Xenotransplant Rejection

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or injury” or “CAI” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerosis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “xenotransplant rejection” encompasses both acute and chronic transplant rejection. The term xenotransplant refers to a transplant where the recipient is a different species than the donor. In particular, the recipient may be human and the donor may be a pig. The term “transplant injury” refers to all manners of graft dysfunction, irrespective of pathological diagnosis. The term “organ injury” refers to biomarkers that track with poor function of the organ, irrespective of the organ being native or a transplant, and irrespective of the etiology.

In some embodiments, the method comprises universal amplification of the extracted RNA. In some embodiments, the universal amplification preferentially amplifies donor-derived RNA over recipient-derived RNA.

In some embodiments, the transplant recipient is a mammal. In some embodiments, the transplant recipient is a human.

In some embodiments, the transplant recipient has received a transplant selected from organ transplant, tissue transplant, cell transplant, and fluid transplant. In some embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, and blood transfusion. In some embodiments, the transplant recipient has received SPK transplant.

In some embodiments, the method further comprises detecting the occurrence or likely occurrence of active rejection of transplantation using the quantified amount of donor-derived RNA. In some embodiments, the method is performed without prior knowledge of donor genotypes.

In some embodiments, each primer pair is designed to amplify a target sequence of about 50-100 bp. In some embodiments, each primer pair is designed to amplify a target sequence of no more than 75 bp. In some embodiments, each primer pair is designed to amplify a target sequence of about 60-75 bp. In some embodiments, each primer pair is designed to amplify a target sequence of about 65 bp.

In some embodiments, the targeted amplification comprises amplifying at least 1,000 polymorphic loci in a single reaction volume. In some embodiments, the targeted amplification comprises amplifying at least 2,000 polymorphic loci in a single reaction volume. In some embodiments, the targeted amplification comprises amplifying at least 5,000 polymorphic loci in a single reaction volume. In some embodiments, the targeted amplification comprises amplifying at least 10,000 polymorphic loci in a single reaction volume. In some embodiments, the targeted amplification comprises amplifying from about 100 to about 50000 polymorphic loci in a single reaction volume. In some embodiments, the targeted amplification comprises amplifying from about 1000 to about 50000 polymorphic loci in a single reaction volume. In some embodiments, the targeted amplification comprises amplifying from about 5000 to about 50000 polymorphic loci in a single reaction volume.

In some embodiments, method further comprises measuring an amount of one or more alleles at the target loci that are polymorphic loci. In some embodiments, the polymorphic loci and the non-polymorphic loci are amplified in a single reaction.

In some embodiments, the quantifying step comprises detecting the amplified target loci using a microarray. In some embodiments, the quantifying step does not comprise using a microarray.

In some embodiments, the targeted amplification comprises simultaneously amplifying 50-50,000 target loci in a single reaction volume using (i) at least 50-50,000 different primer pairs, or (ii) at least 50-50,000 target-specific primers and a universal or tag-specific primer 50-50,000 primer pairs.

In a further aspect, the present invention relates to a method of determining the likelihood of transplant rejection within a transplant recipient, the method comprising: extracting RNA from the blood sample of the transplant recipient, wherein the RNA comprises donor-derived RNA and recipient-derived RNA; performing universal amplification of the extracted RNA; performing targeted amplification at 50-50,000 target loci in a single reaction volume using 50-50,000 primer pairs, wherein the target loci comprise polymorphic loci and non-polymorphic loci; sequencing the amplification products by high-throughput sequencing; and quantifying the amount of donor-derived RNA in the blood sample, wherein a greater amount of dd-RNA indicates a greater likelihood of transplant rejection.

In a further aspect, the present invention relates to a method of diagnosing a transplant within a xenotransplant recipient as undergoing acute rejection of the xenotransplant, the method comprising: extracting RNA from the blood sample of the xenotransplant recipient, wherein the RNA comprises donor-derived RNA and recipient-derived RNA; performing universal amplification of the extracted RNA; performing targeted amplification at 50-50,000 target loci in a single reaction volume using 50-50,000 primer pairs, wherein the target loci comprise polymorphic loci and non-polymorphic loci; sequencing the amplification products by high-throughput sequencing; and quantifying the amount of donor-derived RNA in the blood sample, wherein an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the xenotransplant is undergoing acute rejection.

In some embodiments, the xenotransplant rejection is antibody mediated transplant rejection. In some embodiments, the transplant rejection is T cell mediated transplant rejection.

In some embodiments, an amount of dd-RNA of less than 1% (or 0.9 %, or 0.8%, or 0.7%, or 0.6%, or 0.5%) indicates that the xenotransplant is either undergoing borderline rejection, undergoing other injury, or stable.

In a further aspect, the present invention relates to a method of monitoring immunosuppressive therapy in a subject, the method comprising: extracting RNA from the blood sample of the xenotransplant recipient, wherein the RNA comprises donor-derived RNA and recipient-derived RNA; performing universal amplification of the extracted RNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived RNA in the blood sample, wherein a change in levels of dd-RNA over a time interval is indicative of xenotransplant status.

In some embodiments, the method further comprising adjusting immunosuppressive therapy based on the levels of dd-RNA over the time interval.

In some embodiments, an increase in the levels of dd-RNA is indicative of transplant rejection and a need for adjusting immunosuppressive therapy. In some embodiments, no change or a decrease in the levels of dd-RNA indicates transplant tolerance or stability, and a need for adjusting immunosuppressive therapy.

In some embodiments, an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection. In some embodiments, the transplant rejection is antibody mediated transplant rejection. In some embodiments, the transplant rejection is T cell mediated transplant rejection.

In one aspect the present disclosure relates to a method of administrating immunosuppressive therapy in a xenotransplant recipient, comprising: (a) measuring the amount of cell-free DNA in a blood, plasma, serum, or urine sample of the transplant recipient; and (b) measuring the total amount of donor-derived cell-free DNA in a blood, plasma, serum, or urine sample of the transplant recipient; and (c) titrating the dosage of an immunosuppressive therapy according to the amount of cell-free DNA or a function thereof and the amount of donor-derived cell-free DNA or a function thereof.

In some embodiments, the amount of donor-derived cell-free DNA is measured by: extracting cell-free DNA from the blood, plasma, serum or urine sample of the transplant recipient, wherein the extracted cell-free DNA comprises donor-derived cell-free DNA and recipient-derived cell-free DNA; performing targeted amplification of the extracted DNA at 200-50,000 target loci in a single reaction volume; sequencing the amplified DNA by high-throughput sequencing to obtain sequencing reads and quantifying the amount of donor-derived cell-free DNA based on the sequencing reads.

In some embodiments, the method herein further comprises repeating steps (a)-(b) longitudinally for the same transplant recipient, and determining a longitudinal change in the amount of cell-free DNA or a function thereof and a longitudinal change in the amount of donor-derived cell-free DNA or a function thereof.

In some embodiments, the method herein further comprises titrating the dosage of the immunosuppressive therapy according to the longitudinal change in the total amount of cell-free DNA or a function thereof and the longitudinal change in the amount of donor-derived cell-free DNA or a function thereof.

In one aspect the present disclosure relates to a method of administrating immunosuppressive therapy in a xenotransplant recipient, comprising: (a) measuring the amount of RNA in a blood, plasma, serum, or urine sample of the transplant recipient; and (b) measuring the amount of donor-derived RNA in a blood, plasma, serum, or urine sample of the transplant recipient; and (c) titrating the dosage of an immunosuppressive therapy according to the amount of cell-free DNA or a function thereof and the amount of donor-derived RNA or a function thereof.

In some embodiments, the amount of donor-derived RNA is measured by: extracting RNA from the blood, plasma, serum or urine sample of the transplant recipient, wherein the extracted RNA comprises donor-derived RNA and recipient-derived RNA; preparing a composition of amplified complimentary DNA (cDNA) derived from the extracted RNA by performing multiplex targeted amplification of the cDNA at 200-50,000 animal target loci in a single reaction volume to detect and quantify the amount of animal donor-derived RNA; sequencing the amplified cDNA by high-throughput sequencing to obtain sequencing reads and quantifying the amount of donor-derived RNA based on the sequencing reads.

In some embodiments, the method herein further comprises repeating steps (a)-(b) longitudinally for the same transplant recipient, and determining a longitudinal change in the amount of RNA or a function thereof and a longitudinal change in the amount of donor-derived RNA or a function thereof.

In some embodiments, the method herein further comprises titrating the dosage of the immunosuppressive therapy according to the longitudinal change in the total amount of RNA or a function thereof and the longitudinal change in the amount of donor-derived cell-free DNA or a function thereof.

In some embodiments, an amount of dd-RNA of less than 1% (or 0.9 %, or 0.8%, or 0.7%, or 0.6%, or 0.5%) indicates that the transplant is either undergoing borderline rejection, undergoing other injury, or stable.

In some embodiments, the method does not comprise genotyping the transplant donor and/or the transplant recipient.

In some embodiments, the method further comprises measuring an amount of one or more alleles at the target loci that are polymorphic loci.

In some embodiments, the xenotransplant recipient is a human. In some embodiments, the xenotransplant recipient has received a xenotransplant selected from a kidney transplant, liver transplant, pancreas transplant, islet cell transplant, intestinal transplant, heart transplant, lung transplant, bone marrow transplant, heart valve transplant, or a skin transplant. In some embodiments, the xenotransplant recipient has received SPK transplant.

In some embodiments, the extracting step comprises size selection to enrich for donor-derived RNA and reduce the amount of recipient-derived RNA disposed from bursting blood cells.

In some embodiments, the universal amplification step preferentially amplifies donor-derived RNA over recipient-derived RNA that are disposed from bursting blood cells.

In some embodiments, the method comprises longitudinally collecting a plurality of blood samples from the xenotransplant recipient after transplantation, and repeating steps (a) to (e) for each blood sample collected. In some embodiments, the method comprises collecting and analyzing blood samples from the xenotransplant recipient for a time period of about three months, or about six months, or about twelve months, or about eighteen months, or about twenty-four months, etc. In some embodiments, the method comprises collecting blood samples from the xenotransplant recipient at an interval of about one week, or about two weeks, or about three weeks, or about one month, or about two months, or about three months, etc.

In some embodiments, the method has a sensitivity of at least 80%, or at least 85%, or at least 90%, or at least 95%, or at least 98% in identifying acute rejection (AR) over non-AR with a cutoff threshold of 1% dd-RNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-RNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-RNA and a confidence interval of 95%.

In some embodiments, the method has a sensitivity of at least 80%, or at least 85%, or at least 90%, or at least 95%, or at least 98% in identifying AR over normal, stable allografts (STA) with a cutoff threshold of 1% dd-RNA and a confidence interval of 95%.

In some embodiments, the method has a specificity of at least 80%, or at least 85%, or at least 90%, or at least 95%, or at least 98% in identifying AR over STA with a cutoff threshold of 1% dd-RNA and a confidence interval of 95%.

In some embodiments, the method has an AUC of at least 0.8, or 0.85, or at least 0.9, or at least 0.95, or at least 0.98, or at least 0.99 in identifying AR over STA with a cutoff threshold of 1% dd-RNA and a confidence interval of 95%.

In some embodiments, the method has a sensitivity as determined by a limit of blank (LoB) of 0.5% or less, and a limit of detection (LoD) of 0.5% or less. In some embodiments, LoB is 0.23% or less and LoD is 0.29% or less. In some embodiments, the sensitivity is further determined by a limit of quantitation (LoQ). In some embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some embodiments, LoB is equal to or less than 0.04%, LoD is equal to or less than 0.05%, and/or LoQ is equal to the LoD.

In some embodiments, the method has an accuracy as determined by evaluating a linearity value obtained from linear regression analysis of measured donor fractions as a function of the corresponding attempted spike levels, wherein the linearity value is a R2 value, wherein the R2 value is from about 0.98 to about 1.0. In some embodiments, the R2 value is 0.999. In some embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about-0.0001 to about 0.01. In some embodiments, the slope value is approximately 1, and the intercept value is approximately 0.

In some embodiments, the method has a precision as determined by calculating a coefficient of variation (CV), wherein the CV is less than about 10.0%. CV is less than about 6%. In some embodiments, the CV is less than about 4%. In some embodiments, the CV is less than about 2%. In some embodiments, the CV is less than about 1%.

In some embodiments, the AR is antibody-mediated rejection (ABMR). In some embodiments, the AR is T-cell-mediated rejection (TCMR).

Further disclosed herein are methods for detection of xenotransplant donor-derived RNA (dd-RNA) in a sample from a xenotransplant recipient. In some embodiments, in the methods disclosed herein, the xenotransplant recipient is a mammal. In some embodiments, the xenotransplant recipient is a human. In some embodiments, the xenotransplant is from a pig, a primate, a baboon, a cow, or a dog.

In some embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some embodiments, the transplant recipient has received SPK transplant. In some embodiments, the method may be performed on transplant recipients the day of or after transplant surgery, up to a year following transplant surgery.

In some embodiments, disclosed herein is a method of amplifying target loci of donor-derived RNA (dd-RNA) from a blood sample of a transplant recipient, the method comprising: a) extracting RNA from the blood sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; and c) amplifying the target loci.

In some embodiments, disclosed herein is a method of detecting donor-derived RNA (dd-RNA) in a sample from a transplant recipient, the method comprising: a) extracting RNA from the sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting RNA in the blood sample. In some embodiments, the probes are labelled with a detectable marker.

In some embodiments, disclosed herein is a method of determining the likelihood of transplant rejection within a transplant recipient, the method comprising: a) extracting RNA from the sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; and d) measuring an amount of transplant RNA and an amount of recipient RNA in the recipient sample; wherein a greater amount of dd-RNA indicates a greater likelihood of transplant rejection.

In some embodiments, disclosed herein is a method of diagnosing a transplant within a transplant recipient as undergoing acute rejection, the method comprising: a) extracting RNA from the blood sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; and d) measuring an amount of transplant RNA and an amount of recipient RNA in the recipient sample; wherein an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some embodiments, in the methods disclosed herein, the transplant rejection is antibody mediated transplant rejection. In some embodiments, the transplant rejection is T cell mediated transplant rejection. In some embodiments, an amount of dd-RNA of less than 1% (or 0.9 %, or 0.8%, or 0.7%, or 0.6%, or 0.5%) indicates that the transplant is either undergoing borderline rejection, undergoing other injury, or stable.

In some embodiments, disclosed herein is a method of monitoring immunosuppressive therapy in a subject, the method comprising a) extracting RNA from the blood sample of the transplant recipient, wherein the RNA comprises RNA derived from both the transplanted cells and from the xenotransplant recipient, b) enriching the extracted RNA at target loci, wherein the target loci comprise 50 to 5000 target loci comprising polymorphic loci and non-polymorphic loci; c) amplifying the target loci; and d) measuring an amount of xenotransplant DNA and an amount of recipient DNA in the recipient blood sample; wherein a change in levels of dd-RNA over a time interval is indicative of transplant status. In some embodiments, the method further comprises adjusting immunosuppressive therapy based on the levels of dd-RNA over the time interval. In some embodiments, an increase in the levels of dd-RNA are indicative of transplant rejection and a need for adjusting immunosuppressive therapy. In some embodiments, a change or a decrease in the levels of dd-RNA indicates transplant tolerance or stability, and a need for adjusting immunosuppressive therapy.

In some embodiments, in the methods disclosed herein, the target loci that are amplified in amplicons of about 50-100 bp in length, or about 60-80 bp in length. In some embodiments, the amplicons are about 65 bp in length.

In some embodiments, the methods disclosed herein further comprise measuring an amount of transplant RNA and an amount of recipient RNA in the recipient blood sample.

In some embodiments, the methods disclosed herein do not comprise genotyping the xenotransplant donor and the xenotransplant recipient.

In some embodiments, the methods disclosed herein further comprise detecting the amplified target loci using a microarray.

In some embodiments, in the methods disclosed herein, the polymorphic loci and the non-polymorphic loci are amplified in a single reaction.

In some embodiments, in the methods disclosed herein, the RNA is preferentially enriched at the target loci.

In some embodiments, preferentially enriching the RNA in the sample at the plurality of target loci includes obtaining a plurality of pre-circularized probes where each probe targets one of the target loci, and where the 3′and 5′end of the probes are designed to hybridize to a region of RNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20,21 to 25, 26 to 30, 31 to 60, or a combination thereof, hybridizing the pre-circularized probes to RNA converted to cDNA from the sample, filling the gap between the hybridized probe ends using DNA polymerase, circularizing the pre-circularized probe, and amplifying the circularized probe.

In some embodiments, preferentially enriching the RNA at the plurality of polymorphic loci includes obtaining a plurality of ligation-mediated PCR probes where each PCR probe targets one of the polymorphic loci, and where the upstream and downstream PCR probes are designed to hybridize to a region of cDNA derived from the extracted RNA, on one strand of the cDNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the cDNA from the first sample, filling the gap between the ligation-mediated PCR probe ends using DNA polymerase, ligating the ligation-mediated PCR probes, and amplifying the ligated ligation-mediated PCR probes.

In some embodiments, preferentially enriching the RNA at the plurality of target loci includes obtaining a plurality of hybrid capture probes that target specific loci, hybridizing the hybrid capture probes to the RNA in the sample and physically removing some or all of the unhybridized RNA from the first sample of RNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some embodiments, preferentially enriching the RNA or cDNA derived from the RNA at a plurality of polymorphic loci includes obtaining a plurality of inner forward primers where each primer targets one of the polymorphic loci, and where the 3′end of the inner forward primers are designed to hybridize to a region of RNA or cDNA derived from the RNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′end of the inner reverse primers are designed to hybridize to a region of RNA or cDNA derived from the RNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the RNA or cDNA derived from the RNA, and amplifying the cDNA using the polymerase chain reaction to form amplicons.

In some embodiments, the method also includes obtaining a plurality of outer forward primers where each primer targets one of the polymorphic loci, and where the outer forward primers are designed to hybridize to the region of RNA or cDNA derived from the RNA upstream from the inner forward primer, optionally obtaining a plurality of outer reverse primers where each primer targets one of the polymorphic loci, and where the outer reverse primers are designed to hybridize to the region of RNA or cDNA derived from the RNA immediately downstream from the inner reverse primer, hybridizing the first primers to the RNA or cDNA derived from the RNA, and amplifying the cDNA using the polymerase chain reaction.

In some embodiments, the method also includes obtaining a plurality of outer reverse primers where each primer targets one of the polymorphic loci, and where the outer reverse primers are designed to hybridize to the region of RNA or cDNA derived from the RNA immediately downstream from the inner reverse primer, optionally obtaining a plurality of outer forward primers where each primer targets one of the polymorphic loci, and where the outer forward primers are designed to hybridize to the region of RNA or cDNA derived from the RNA upstream from the inner forward primer, hybridizing the first primers to the RNA or cDNA derived from the RNA, and amplifying the RNA or cDNA derived from the RNA using the polymerase chain reaction.

In some embodiments, preparing the first sample further includes appending universal adapters to the RNA in the first sample and amplifying the RNA in the first sample using the polymerase chain reaction. In some embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some embodiments, amplifying the RNA or cDNA derived from the RNA is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some embodiments, preparing the sample further comprises dividing the sample into a plurality of portions, and where the RNA or cDNA derived from the RNA in each portion is preferentially enriched at a subset of the plurality of polymorphic loci. In some embodiments, the inner primers are selected by identifying primer pairs likely to form undesired primer duplexes and removing from the plurality of primers at least one of the pair of primers identified as being likely to form undesired primer duplexes. In some embodiments, the inner primers contain a region that is designed to hybridize either upstream or downstream of the targeted polymorphic locus, and optionally contain a universal priming sequence designed to allow PCR amplification. In some embodiments, at least some of the primers additionally contain a random region that differs for each individual primer molecule. In some embodiments, at least some of the primers additionally contain a molecular barcode.

In some embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some embodiments, the method does not comprise using a microarray.

In some embodiments, the method comprises (a) performing multiplex polymerase chain reaction (PCR) on the RNA (or cDNA) sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some embodiments, the method does not comprise using a microarray.

In some embodiments, the method also includes obtaining genotypic data from one or both of the transplant donor and the transplant recipient. In some embodiments, obtaining genotypic data from one or both of the transplant donor and the transplant recipient includes preparing the RNA from the donor and the recipient where the preparing comprises preferentially enriching the RNA at the plurality of polymorphic loci to give prepared RNA or cDNA, optionally amplifying the prepared cDNA, and measuring the RNA in the prepared sample at the plurality of polymorphic loci.

In some embodiments, building a joint distribution model for the expected allele count probabilities of the plurality of polymorphic loci on the chromosome is done using the obtained genetic data from the one or both of the transplant donor and the transplant recipient. In some embodiments, the first sample has been isolated from transplant recipient plasma and where the obtaining genotypic data from the transplant recipient is done by estimating the recipient genotypic data from the RNA measurements made on the prepared sample.

In some embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some embodiments, the plurality of polymorphic loci are SNPs. In some embodiments, measuring the RNA in the prepared sample is done by sequencing.

In some embodiments, a diagnostic box is disclosed for helping to determine transplant status in a transplant recipient where the diagnostic box is capable of executing the preparing and measuring steps of the disclosed methods.

In some embodiments, the allele counts are probabilistic rather than binary. In some embodiments, measurements of the RNA in the prepared sample at the plurality of polymorphic loci are also used to determine whether or not the transplant has inherited one or a plurality of linked haplotypes.

In some embodiments, building a joint distribution model for allele count probabilities is done by using data about the probability of chromosomes crossing over at different locations in a chromosome to model dependence between polymorphic alleles on the chromosome. In some embodiments, building a joint distribution model for allele counts and the step of determining the relative probability of each hypothesis are done using a method that does not require the use of a reference chromosome.

In some embodiments, determining the relative probability of each hypothesis makes use of an estimated fraction of donor-derived RNA (dd-RNA) in the prepared sample. In some embodiments, the DNA measurements from the prepared sample used in calculating allele count probabilities and determining the relative probability of each hypothesis comprise primary genetic data. In some embodiments, selecting the transplant status corresponding to the hypothesis with the greatest probability is carried out using maximum likelihood estimates or maximum a posteriori estimates.

In some embodiments, calling the transplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some embodiments, a confidence estimate is calculated for the called transplant status. In some embodiments, the method also includes taking a clinical action based on the called transplant status.

In some embodiments, a report displaying a determined transplant status is generated using the method. In some embodiments, a kit is disclosed for determining a transplant status designed to be used with the methods disclosed herein, the kit including a plurality of inner forward primers and optionally the plurality of inner reverse primers, where each of the primers is designed to hybridize to the region of RNA immediately upstream and/or downstream from one of the target sites, where the region of hybridization is separated from the target site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some embodiments, the methods disclosed herein comprise a selection step to select for shorter RNA.

In some embodiments, the methods disclosed herein comprise a universal application step to enrich for RNA.

In some embodiments, the determination that the amount of dd-RNA above a cutoff threshold is indicative of acute rejection of the transplant. Machine learning and artificial intelligence may be used to resolve rejection vs non-rejection.

In some embodiments, the cutoff threshold value is expressed as percentage of dd-RNA (dd-RNA %) in the blood sample.

In some embodiments, the cutoff threshold value is expressed as copy number of dd-RNA per volume unit of the blood sample.

In some embodiments, the cutoff threshold value is expressed as copy number of dd-RNA per volume unit of the blood sample multiplied by body mass or blood volume of the transplant recipient.

In some embodiments, the cutoff threshold value takes into account the body mass or blood volume of the patient.

In some embodiments, the cutoff threshold value takes into account one or more of the followings: donor genome copies per volume of plasma, cell-free DNA or RNA yield per volume of plasma, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some embodiments, the cutoff threshold value is scaled according to the amount of total RNA in the blood sample.

In some embodiments, the method has a sensitivity of at least 80% in identifying acute rejection (AR) over non-AR when the dd-RNA amount is above the cutoff threshold value scaled according to the amount of total RNA in the sample and a confidence interval of 95%.

In some embodiments, the method has a specificity of at least 70% in identifying acute rejection (AR) over non-AR when the dd-RNA amount is above the cutoff threshold value scaled according to the amount of total RNA in the blood sample and a confidence interval of 95%.

In some embodiments, the method has a sensitivity of at least 80% in identifying acute rejection (AR) over non-AR when the dd-RNA amount is above the cutoff threshold value scaled according to the amount of total RNA in the sample and a confidence interval of 95%. In some embodiments, the method has a sensitivity of at least 85% in identifying acute rejection (AR) over non-AR when the dd-RNA amount is above the cutoff threshold value scaled according to the amount of total RNA in the blood sample and a confidence interval of 95%. In some embodiments, the method has a sensitivity of at least 90% in identifying acute rejection (AR) over non-AR when the dd-RNA amount is above the cutoff threshold value scaled according to the amount of total RNA in the blood sample and a confidence interval of 95%. In some embodiments, the method has a sensitivity of at least 95% in identifying acute rejection (AR) over non-AR when the dd-RNA amount is be above the cutoff threshold value scaled according to the amount of total RNA in the sample and a confidence interval of 95%.

In some embodiments, the method has a specificity of at least 70% in identifying acute rejection (AR) over non-AR when the RNA amount is above the cutoff threshold value scaled according to the amount of total RNA in the blood sample and a confidence interval of 95%. In some embodiments, the method has a specificity of at least 75% in identifying acute rejection (AR) over non-AR when the dd-RNA amount is above the cutoff threshold value scaled according to the amount of total RNA in the blood sample and a confidence interval of 95%. In some embodiments, the method has a specificity of at least 85% in identifying acute rejection (AR) over non-AR when the dd-RNA amount is above the cutoff threshold value scaled according to the amount of total RNA in the blood sample and a confidence interval of 95%. In some embodiments, the method has a specificity of at least 90% in identifying acute rejection (AR) over non-AR when the dd-cfDNA amount is above the cutoff threshold value scaled according to the amount of total RNA in the blood sample and a confidence interval of 95%. In some embodiments, the method has a specificity of at least 95% in identifying acute rejection (AR) over non-AR when the dd-RNA amount is above the cutoff threshold value scaled according to the amount of total RNA in the blood sample and a confidence interval of 95%.

### Analysis of Donor-Derived Cell-Free DNA for Monitoring Xenotransplant Rejection

In one aspect, the present invention further comprises: (i) measuring the amount of donor-derived cell-free DNA in a sample obtained from the xenotransplant recipient, extracting cell-free DNA from the sample obtained from the xenotransplant recipient, wherein the extracted cell-free DNA comprises donor-derived cell-free DNA and recipient-derived cell-free DNA; (ii) performing targeted amplification of the extracted DNA at 10-50,000 target loci in a single reaction volume; (iii) sequencing the amplified DNA to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection.

In another aspect, the present invention relates to a method of quantifying the amount of donor-derived cell-free DNA (dd-cfDNA) in a blood sample of a xenotransplant recipient, comprising: extracting DNA from the blood sample of the xenotransplant recipient, wherein the DNA comprises donor-derived cell-free DNA and recipient-derived cell-free DNA; performing targeted amplification at 100-50,000 or 500-50,000 target loci in a single reaction volume using 100-50,000 or 500-50,000 primer pairs, wherein the target loci comprise polymorphic loci and non-polymorphic loci, and wherein each primer pair is designed to amplify a target sequence of no more than 100 bp; and quantifying the amount of donor-derived cell-free DNA in the amplification products.

In another aspect, the present invention relates to a method of quantifying the amount of donor-derived cell-free DNA (dd-cfDNA) in a blood sample of a xenotransplant recipient, comprising: extracting DNA from the blood sample of the xenotransplant recipient, wherein the DNA comprises donor-derived cell-free DNA and recipient-derived cell-free DNA, and wherein the extracting step comprises size selection to enrich for donor-derived cell-free DNA and reduce the amount of recipient-derived cell-free DNA disposed from bursting white-blood cells; performing targeted amplification at 500-50,000 target loci in a single reaction volume using 500-50,000 primer pairs, wherein the target loci comprise polymorphic loci and non-polymorphic loci; and quantifying the amount of donor-derived cell-free DNA in the amplification products.

In another aspect, the present invention relates to a method of detecting donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 100-50,000 target loci in a single reaction volume using 100-50,000 primer pairs, wherein the target loci comprise polymorphic loci and non-polymorphic loci; sequencing the amplification products by high-throughput sequencing; and quantifying the amount of donor-derived cell-free DNA.

In some embodiments, the method further comprises performing universal amplification of the extracted DNA. In some embodiments, the universal amplification preferentially amplifies donor-derived cell-free DNA over recipient-derived cell-free DNA that are disposed from bursting white-blood cells.

In some embodiments, the xenotransplant recipient is a mammal. In some embodiments, the transplant recipient is a human. In some embodiments, the xenotransplant is from a pig, a primate, a baboon, a cow, or a dog.

In some embodiments, the xenotransplant recipient has received a xenotransplant selected from organ transplant, tissue transplant, cell transplant, and fluid transplant. In some embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, and blood transfusion. In some embodiments, the transplant recipient has received SPK transplant.

In some embodiments, the quantifying step comprises determining the percentage of donor-derived cell-free DNA out of the total of donor-derived cell-free DNA and recipient-derived cell-free DNA in the blood sample. In some embodiments, the quantifying step comprises determining the number of copies of donor-derived cell-free DNA per volume unit of the blood sample.

In some embodiments, the method further comprises detecting the occurrence or likely occurrence of active rejection of transplantation using the quantified amount of donor-derived cell-free DNA. In some embodiments, the method is performed without prior knowledge of donor genotypes.

In some embodiments, each primer pair is designed to amplify a target sequence of about 50-100 bp. In some embodiments, each primer pair is designed to amplify a target sequence of no more than 75 bp. In some embodiments, each primer pair is designed to amplify a target sequence of about 60-75 bp. In some embodiments, each primer pair is designed to amplify a target sequence of about 65 bp.

In some embodiments, the targeted amplification comprises amplifying at least 1,000 polymorphic loci in a single reaction volume. In some embodiments, the targeted amplification comprises amplifying at least 2,000 polymorphic loci in a single reaction volume. In some embodiments, the targeted amplification comprises amplifying at least 5,000 polymorphic loci in a single reaction volume. In some embodiments, the targeted amplification comprises amplifying at least 10,000 polymorphic loci in a single reaction volume. In some embodiments, the targeted amplification comprises amplifying 10 to 10,000, 10 to 50,000, 100 to 50,000, or 1000 to 50,000 polymorphic loci in a single reaction volume.

In some embodiments, method further comprises measuring an amount of one or more alleles at the target loci that are polymorphic loci. In some embodiments, the polymorphic loci and the non-polymorphic loci are amplified in a single reaction.

In some embodiments, the quantifying step comprises detecting the amplified target loci using a microarray. In some embodiments, the quantifying step does not comprise using a microarray.

In some embodiments, the targeted amplification comprises simultaneously amplifying 500-50,000 target loci in a single reaction volume using (i) at least 500-50,000 different primer pairs, or (ii) at least 500-50,000 target-specific primers and a universal or tag-specific primer 500-50,000 primer pairs.

In a further aspect, the present invention relates to a method of determining the likelihood of transplant rejection within a transplant recipient, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a greater amount of dd-cfDNA indicates a greater likelihood of transplant rejection.

In a further aspect, the present invention relates to a method of diagnosing a xenotransplant within a xenotransplant recipient as undergoing acute rejection, the method comprising: extracting DNA from the blood sample of the xenotransplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the xenotransplant is undergoing acute rejection.

In some embodiments, the xenotransplant rejection is antibody mediated transplant rejection. In some embodiments, the xenotransplant rejection is T cell mediated transplant rejection.

In some embodiments, an amount of dd-cfDNA of less than 1% (or 0.9 %, or 0.8%, or 0.7%, or 0.6%, or 0.5%) indicates that the xenotransplant is either undergoing borderline rejection, undergoing other injury, or stable.

In a further aspect, the present invention relates to a method of monitoring immunosuppressive therapy in a subject, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a change in levels of dd-cfDNA over a time interval is indicative of xenotransplant status.

In some embodiments, the method further comprising adjusting immunosuppressive therapy based on the levels of dd-cfDNA over the time interval.

In some embodiments, an increase in the levels of dd-cfDNA is indicative of xenotransplant rejection and a need for adjusting immunosuppressive therapy. In some embodiments, no change or a decrease in the levels of dd-cfDNA indicates xenotransplant tolerance or stability, and a need for adjusting immunosuppressive therapy.

In some embodiments, an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the xenotransplant is undergoing acute rejection. In some embodiments, the xenotransplant rejection is antibody mediated transplant rejection. In some embodiments, the xenotransplant rejection is T cell mediated transplant rejection.

In some embodiments, an amount of dd-cfDNA of less than 1% (or 0.9 %, or 0.8%, or 0.7%, or 0.6%, or 0.5%) indicates that the xenotransplant is either undergoing borderline rejection, undergoing other injury, or stable.

In some embodiments, the method does not comprise genotyping the transplant donor and/or the transplant recipient.

In some embodiments, the method further comprises measuring an amount of one or more alleles at the target loci that are polymorphic loci.

In some embodiments, the target loci comprise at least 1,000 polymorphic loci, or at least 2,000 polymorphic loci, or at least 5,000 polymorphic loci, or at least 10,000 polymorphic loci.

In some embodiments, the target loci that are amplified in amplicons of about 50-100 bp in length, or about 50-90 bp in length, or about 60-80 bp in length, or about 60-75 bp in length, or about 65 bp in length.

In some embodiments, the xenotransplant recipient is a human. In some embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some embodiments, the transplant recipient has received SPK transplant.

In some embodiments, the extracting step comprises size selection to enrich for donor-derived cell-free DNA and reduce the amount of recipient-derived cell-free DNA disposed from bursting white-blood cells.

In some embodiments, the universal amplification step preferentially amplifies donor-derived cell-free DNA over recipient-derived cell-free DNA that are disposed from bursting white-blood cells.

In some embodiments, the method comprises longitudinally collecting a plurality of blood samples from the transplant recipient after transplantation, and repeating steps (a) to (e) for each blood sample collected. In some embodiments, the method comprises collecting and analyzing blood samples from the transplant recipient for a time period of about three months, or about six months, or about twelve months, or about eighteen months, or about twenty-four months, etc. In some embodiments, the method comprises collecting blood samples from the transplant recipient at an interval of about one week, or about two weeks, or about three weeks, or about one month, or about two months, or about three months, etc.

In some embodiments, the method has a sensitivity of at least 80%, or at least 85%, or at least 90%, or at least 95%, or at least 98% in identifying acute rejection (AR) over non-AR with a cutoff threshold of 1% dd-cfDNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-cfDNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-cfDNA and a confidence interval of 95%.

In some embodiments, the method has a sensitivity of at least 80%, or at least 85%, or at least 90%, or at least 95%, or at least 98% in identifying AR over normal, stable allografts (STA) with a cutoff threshold of 1% dd-cfDNA and a confidence interval of 95%.

In some embodiments, the method has a specificity of at least 80%, or at least 85%, or at least 90%, or at least 95%, or at least 98% in identifying AR over STA with a cutoff threshold of 1% dd-cfDNA and a confidence interval of 95%.

In some embodiments, the method has an AUC of at least 0.8, or 0.85, or at least 0.9, or at least 0.95, or at least 0.98, or at least 0.99 in identifying AR over STA with a cutoff threshold of 1% dd-cfDNA and a confidence interval of 95%.

In some embodiments, the method has a sensitivity as determined by a limit of blank (LoB) of 0.5% or less, and a limit of detection (LoD) of 0.5% or less. In some embodiments, LoB is 0.23% or less and LoD is 0.29% or less. In some embodiments, the sensitivity is further determined by a limit of quantitation (LoQ). In some embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some embodiments, LoB is equal to or less than 0.04%, LoD is equal to or less than 0.05%, and/or LoQ is equal to the LoD.

In some embodiments, the method has an accuracy as determined by evaluating a linearity value obtained from linear regression analysis of measured donor fractions as a function of the corresponding attempted spike levels, wherein the linearity value is a R2 value, wherein the R2 value is from about 0.98 to about 1.0. In some embodiments, the R2 value is 0.999. In some embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about-0.0001 to about 0.01. In some embodiments, the slope value is approximately 1, and the intercept value is approximately 0.

In some embodiments, the method has a precision as determined by calculating a coefficient of variation (CV), wherein the CV is less than about 10.0%. CV is less than about 6%. In some embodiments, the CV is less than about 4%. In some embodiments, the CV is less than about 2%. In some embodiments, the CV is less than about 1%.

In some embodiments, the AR is antibody-mediated rejection (ABMR). In some embodiments, the AR is T-cell-mediated rejection (TCMR).

Further disclosed herein are methods for detection of transplant donor-derived cell-free DNA (dd-cfDNA) in a sample from a xenotransplant recipient. In some embodiments, in the methods disclosed herein, the xenotransplant recipient is a mammal. In some embodiments, the xenotransplant recipient is a human. In some embodiments, the xenotransplant recipient has received a xenotransplant selected from a kidney transplant, liver transplant, pancreas transplant, islet cell transplant, intestinal transplant, heart transplant, lung transplant, bone marrow transplant, heart valve transplant, or a skin transplant. In some embodiments, the xenotransplant recipient has received SPK transplant. In some embodiments, the method may be performed on xenotransplant recipients the day of or after transplant surgery, up to a year following transplant surgery.

In some embodiments, disclosed herein is a method of amplifying target loci of donor-derived cell-free DNA (dd-cfDNA) from a blood sample of a xenotransplant recipient, the method comprising: a) extracting DNA from the blood sample of the xenotransplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; and c) amplifying the target loci.

In some embodiments, disclosed herein is a method of detecting donor-derived cell-free DNA (dd-cfDNA) in a blood sample from a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting dd-cfDNA in the blood sample. In some embodiments, the probes are labelled with a detectable marker.

In some embodiments, disclosed herein is a method of determining the likelihood of xenotransplant rejection within a transplant recipient, the method comprising: a) extracting DNA from the blood sample of the xenotransplant recipient, wherein the DNA comprises cell-free DNA derived from both the transplanted cells and from the xenotransplant recipient, b) enriching the extracted DNA at target loci, wherein the target loci comprise 50 to 5000 target loci comprising polymorphic loci and non-polymorphic loci; c) amplifying the target loci; and d) measuring an amount of xenotransplant DNA and an amount of xenotransplant recipient DNA in the recipient blood sample; wherein a greater amount of dd-cfDNA indicates a greater likelihood of xenotransplant rejection.

In some embodiments, disclosed herein is a method of diagnosing a xenotransplant within a xenotransplant recipient as undergoing acute rejection, the method comprising: a) extracting DNA from the blood sample of the transplant recipient, wherein the DNA comprises cell-free DNA derived from both the transplanted cells and from the xenotransplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the xenotransplant is undergoing acute rejection.

In some embodiments, in the methods disclosed herein, the xenotransplant rejection is antibody mediated transplant rejection. In some embodiments, the xenotransplant rejection is T cell mediated transplant rejection. In some embodiments, an amount of dd-cfDNA of less than 1% (or 0.9 %, or 0.8%, or 0.7%, or 0.6%, or 0.5%) indicates that the xenotransplant is either undergoing borderline rejection, undergoing other injury, or stable.

In some embodiments, disclosed herein is a method of monitoring immunosuppressive therapy in a subject, the method comprising a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a change in levels of dd-cfDNA over a time interval is indicative of xenotransplant status. In some embodiments, the method further comprises adjusting immunosuppressive therapy based on the levels of dd-cfDNA over the time interval. In some embodiments, an increase in the levels of dd-cfDNA are indicative of xenotransplant rejection and a need for adjusting immunosuppressive therapy. In some embodiments, a change or a decrease in the levels of dd-cfDNA indicates xenotransplant tolerance or stability, and a need for adjusting immunosuppressive therapy.

In some embodiments, in the methods disclosed herein, the target loci that are amplified in amplicons of about 50-100 bp in length, or about 60-80 bp in length. In some embodiments, the amplicons are about 65 bp in length.

### Analytical Methods

In some embodiments, the method also includes obtaining genotypic data from one or both of the xenotransplant donor and the xenotransplant recipient. In some embodiments, obtaining genotypic data from one or both of the xenotransplant donor and the xenotransplant recipient includes preparing the DNA from the donor and the recipient where the preparing comprises preferentially enriching the DNA at the plurality of polymorphic loci to give prepared DNA, optionally amplifying the prepared DNA, and measuring the DNA in the prepared sample at the plurality of polymorphic loci.

In some embodiments, building a joint distribution model for the expected allele count probabilities of the plurality of polymorphic loci on the chromosome is done using the obtained genetic data from the one or both of the transplant donor and the transplant recipient. In some embodiments, the first sample has been isolated from transplant recipient plasma and where the obtaining genotypic data from the transplant recipient is done by estimating the recipient genotypic data from the DNA measurements made on the prepared sample.

In some embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some embodiments, the plurality of polymorphic loci are SNPs. In some embodiments, measuring the DNA or RNA in the prepared sample is done by sequencing.

In some embodiments, a diagnostic box is disclosed for helping to determine xenotransplant status in a xenotransplant recipient where the diagnostic box is capable of executing the preparing and measuring steps of the disclosed methods.

In some embodiments, the allele counts are probabilistic rather than binary. In some embodiments, measurements of the DNA in the prepared sample at the plurality of polymorphic loci are also used to determine whether or not the xenotransplant has inherited one or a plurality of linked haplotypes.

In some embodiments, building a joint distribution model for allele count probabilities is done by using data about the probability of chromosomes crossing over at different locations in a chromosome to model dependence between polymorphic alleles on the chromosome. In some embodiments, building a joint distribution model for allele counts and the step of determining the relative probability of each hypothesis are done using a method that does not require the use of a reference chromosome.

In some embodiments, determining the relative probability of each hypothesis makes use of an estimated fraction of donor-derived RNA and/or the donor-derived cell-free DNA (dd-cfDNA) in the prepared sample. In some embodiments, the DNA measurements from the prepared sample used in calculating allele count probabilities and determining the relative probability of each hypothesis comprise primary genetic data. In some embodiments, selecting the transplant status corresponding to the hypothesis with the greatest probability is carried out using maximum likelihood estimates or maximum a posteriori estimates.

In some embodiments, calling the xenotransplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some embodiments, a confidence estimate is calculated for the called xenotransplant status. In some embodiments, the method also includes taking a clinical action based on the called xenotransplant status.

In some embodiments, a report displaying a determined xenotransplant status is generated using the method. In some embodiments, a kit is disclosed for determining a xenotransplant status designed to be used with the methods disclosed herein, the kit including a plurality of inner forward primers and optionally the plurality of inner reverse primers, where each of the primers is designed to hybridize to the region of DNA immediately upstream and/or downstream from one of the polymorphic sites on the target chromosome, and optionally additional chromosomes, where the region of hybridization is separated from the polymorphic site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some embodiments, the cutoff threshold value takes into account one or more of the followings: donor genome copies per volume of plasma, cell-free DNA yield per volume of plasma, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some embodiments, the cutoff threshold value is scaled according to the amount of total cfDNA in the blood sample.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled according to the amount of total cfDNA in the blood sample and a confidence interval of 95%.

### Multiplex Amplification

In some embodiments, the method comprises performing a multiplex amplification reaction to amplify a plurality of target loci in one reaction mixture before determining the sequences of the selectively enriched RNA or DNA.

In certain illustrative embodiments, the nucleic acid sequence data is generated by performing high throughput RNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 100; 200; 500; 1,000; 2,000; 5,000; 10,000;20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to at least 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify cfDNA obtained from a blood, plasma, serum, solid tissue, or urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (˜10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some embodiments, the sequences of the amplicons are determined by performing high-throughput sequencing.

The genetic data of the transplant recipient and/or of the transplant donor can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, PacBio, Oxford Nanopore, or any other sequencing method. In some embodiments, the high throughput sequencing is performed on Illumina NEXTSEQ®. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that arc fluorescing due to proper amplification. This positive fraction accurately indicates the initial amount of template nucleic acid.

### Tracer DNA and Use Thereof

Tracer DNA for estimating the amount of total cfDNA in a sample is described in US Prov. Appl. No. 63/031,879 filed May 29, 2020 and titled “Improved Methods for Detection of Donor Derived Cell-Free DNA”, which is incorporated herein by reference in its entirety. In some embodiments, the Tracer DNA comprises synthetic double-stranded DNA molecules. In some embodiments, the Tracer DNA comprises DNA molecules of non-human origin.

In some embodiments, the Tracer DNA comprises DNA molecules having a length of about 50-500 bp, or about 75-300 bp, or about 100-250 bp, or about 125-200 bp, or about 125 bp, or about 160 bp, or about 200 bp, or about 500-1,000 bp.

In some embodiments, the Tracer DNA comprises DNA molecules having the same or substantially the same length, such as a DNA molecule having a length of about 125 bp, or about 160 bp, or about 200 bp. In some embodiments, the Tracer DNA comprises DNA molecules having different lengths, such as a first DNA molecule having a length of about 125 bp, a second DNA molecule having a length of about 160 bp, and a third DNA molecule having a length of about 200 bp. In some embodiments, the DNA molecules having different lengths are used to determine size distribution of the cell-free DNA in the sample

In some embodiments, the Tracer DNA comprises a target sequence, wherein the target sequence comprises a barcode positioned between a pair of primer binding sites capable of binding to a pair of primers. In some embodiments, at least part of the Tracer DNA is designed based on an endogenous human SNP locus, by replacing an endogenous sequence containing the SNP locus with the barcode. During the mmPCR target enrichment step, the primer pair targeting the SNP locus can also amplify the portion of Tracer DNA containing the barcode.

In some embodiments, the barcode is an arbitrary barcode. In some embodiments, the barcode comprises reverse complement of a corresponding endogenous genome sequence capable of being amplified by the same primer pair.

In some embodiments, the target sequence within the Tracer DNA is flanked on one or both sides by endogenous genome sequences. In some embodiments, the target sequence within the Tracer DNA is flanked on one or both sides by non-endogenous sequences.

In some embodiments, the Tracer DNA comprises a plurality of target sequences. In some embodiments, the Tracer DNA comprises a first target sequence comprising a first barcode positioned between a first pair of primer binding sites capable of binding to a first pair of primers, and a second barcode positioned between a second pair of primer binding sites capable of binding to a second pair of primers. In some embodiments, the first and/or second target sequence is designed based on one or more endogenous human SNP loci, by replacing an endogenous sequence containing a SNP locus with a barcode. In some embodiments, the first and/or second barcode is an arbitrary barcode. In some embodiments, the first and/or second barcode comprises reverse complement of a corresponding endogenous genome sequence capable of being amplified by the first or second primer pair. In some embodiments, the first and/or second target sequence within the Tracer DNA is flanked on one or both sides by endogenous genome sequences. In some embodiments, the first and/or second target sequence within the Tracer DNA is flanked on one or both sides by non-endogenous sequences.

In some embodiments, the Tracer DNA comprises DNA molecules having the same or substantially the same sequence. In some embodiments, the Tracer DNA comprises DNA molecules having different sequences.

In some embodiments, the Tracer DNA comprises a first DNA comprising a first target sequence and a second DNA comprising a second target sequence. In some embodiments, the first target sequence and second target sequence have different barcodes positioned between the same primer binding sites. In some embodiments, the first target sequence and second target sequence have different barcodes positioned between the same primer binding sites, wherein the different barcodes have the same or substantially the same lengths. In some embodiments, the first target sequence and second target sequence have different barcodes positioned between the same primer binding sites, wherein the different barcodes have different lengths. In some embodiments, the first target sequence and second target sequence are designed based on different endogenous human SNP loci, and hence comprise different primer binding sites. In some embodiments, the amount of first DNA and the amount of the second DNA are the same or substantially the same in the Tracer DNA. In some embodiments, the amount of first DNA and the amount of the second DNA are different in the Tracer DNA.

In certain embodiments, Tracer DNA can be used to improve accuracy and precision of the method described herein, help quantify over a wider input range, assess efficiency of different steps at different size ranges, and/or calculate fragment size-distribution of input material.

Some embodiments of the present invention relate to a method of quantifying the amount of total cell-free DNA in a biological sample, comprising: a) isolating cell-free DNA from the biological sample, wherein a first Tracer DNA is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA.

In some embodiments, the method comprises adding the first Tracer DNA to a whole blood sample before plasma extraction. In some embodiments, the method comprises adding the first Tracer DNA to a plasma sample after plasma extraction and before isolation of the cell-free DNA. In some embodiments, the method comprises adding the first Tracer DNA to a composition comprising the isolated cell-free DNA. In some embodiments, the method comprises ligating adaptors to the isolated cell-free DNA to obtain a composition comprising adaptor-ligated DNA, and adding the first Tracer DNA to the composition comprising adaptor-ligated DNA.

In some embodiments, the method further comprises adding a second Tracer DNA before the targeted amplification. In some embodiments, the method further comprises adding a second Tracer DNA after the targeted amplification.

In some embodiments, the amount of total cfDNA in the sample is estimated using the NOR of the Tracer DNA (identifiable by the barcode), the NOR of sample DNA, and the known amount of the Tracer DNA added to the plasma sample. In some embodiments, the ratio between the NOR of the Tracer DNA and the NOR of sample DNA is used to quantify the amount of total cell-free DNA. In some embodiments, the ratio between the NOR of the barcode and the NOR of the corresponding endogenous genome sequence is used to quantify the amount of total cell-free DNA. In some embodiments, this information along with the plasma volume can also be used to calculate the amount of cfDNA per volume of plasma. In some embodiments, these can be multiplied by the percentage of donor DNA to calculate the total donor cfDNA and the donor cfDNA per volume of plasma.

Accordingly, in another aspect, the present invention relates to a method of quantifying the amount of total cell-free DNA in a biological sample, comprising: a) isolating cell-free DNA from the biological sample, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA composition.

In further aspect, the present invention relates to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

In a further aspect, the present invention relates to a method of determining the occurrence or likely occurrence of transplant rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of total cell-free DNA is quantified using sequencing reads derived from the first Tracer DNA composition, and determining the occurrence or likely occurrence of transplant rejection using the amount of donor-derived cell-free DNA by comparing the amount of donor-derived cell-free DNA to a threshold value, wherein the threshold value is determined according to the amount of total cell-free DNA.

In some embodiments, the threshold value is a function of the number of sequencing reads of the donor-derived cell-free DNA.

In some embodiments, the method further comprises flagging the sample if the amount of total cell-free DNA falls outside a pre-determined range. In some embodiments, the method further comprises flagging the sample if the amount of total cell-free DNA is above a pre-determined value. In some embodiments, the method further comprises flagging the sample if the amount of total cell-free DNA is below a pre-determined value.

In some embodiments, the method comprises adding the first Tracer DNA composition to a whole blood sample before plasma extraction. In some embodiments, the method comprises adding the first Tracer DNA composition to a plasma sample after plasma extraction and before isolation of the cell-free DNA. In some embodiments, the method comprises adding the first Tracer DNA composition to a composition comprising the isolated cell-free DNA. In some embodiments, the method comprises ligating adaptors to the isolated cell-free DNA to obtain a composition comprising adaptor-ligated DNA, and adding the first Tracer DNA composition to the composition comprising adaptor-ligated DNA.

In some embodiments, the method further comprises adding a second Tracer DNA composition before the targeted amplification. In some embodiments, the method further comprises adding a second Tracer DNA composition after the targeted amplification.

In some embodiments, the first and/or second Tracer DNA composition comprises a plurality of DNA molecules having different sequences.

In some embodiments, the first and/or second Tracer DNA composition comprises a plurality of DNA molecules having at different concentrations.

In some embodiments, the first and/or second Tracer DNA composition comprises a plurality of DNA molecules having different lengths. In some embodiments, the plurality of DNA molecules having different lengths are used to determine size distribution of the cell-free DNA in the sample.

In some embodiments, the first and/or second Tracer DNA composition comprises a plurality of DNA molecules of non-human origin.

In some embodiments, the first and/or second Tracer DNA composition each comprises a target sequence, wherein the target sequence comprises a barcode positioned between a pair of primer binding sites capable of binding to one of the primer pairs. In some embodiments, the barcode comprises reverse complement of a corresponding endogenous genome sequence capable of being amplified by the same primer pair.

In some embodiments, the ratio between the number of reads of the Tracer DNA and the number of reads of sample DNA is used to quantify the amount of total cell-free DNA. In some embodiments, the ratio between the number of reads of the barcode and the number of reads of the corresponding endogenous genome sequence is used to quantify the amount of total cell-free DNA.

In some embodiments, the target sequence is flanked on one or both sides by endogenous genome sequences. In some embodiments, the target sequence is flanked on one or both sides by non-endogenous sequences.

In some embodiments, the first and/or second Tracer DNA composition comprises synthetic double-stranded DNA molecules. In some embodiments, the first and/or second Tracer DNA composition comprises DNA molecules having a length of 50-500 bp. In some embodiments, the first and/or second Tracer DNA composition comprises DNA molecules having a length of 75-300 bp. In some embodiments, the first and/or second Tracer DNA composition comprises DNA molecules having a length of 100-250 bp. In some embodiments, the first and/or second Tracer DNA composition comprises DNA molecules having a length of 125-200 bp. In some embodiments, the first and/or second Tracer DNA composition comprises DNA molecules having a length of about 200 bp. In some embodiments, the first and/or second Tracer DNA composition comprises DNA molecules having a length of about 160 bp. In some embodiments, the first and/or second Tracer DNA composition comprises DNA molecules having a length of about 125 bp. In some embodiments, the first and/or second Tracer DNA composition comprises DNA molecules having a length of 500-1,000 bp.

In some embodiments, the targeted amplification comprises amplifying at least 100 polymorphic or SNP loci in a single reaction volume. In some embodiments, the targeted amplification comprises amplifying at least 200 polymorphic or SNP loci in a single reaction volume. In some embodiments, the targeted amplification comprises amplifying at least 500 polymorphic or SNP loci in a single reaction volume. In some embodiments, the targeted amplification comprises amplifying at least 1,000 polymorphic or SNP loci in a single reaction volume. In some embodiments, the targeted amplification comprises amplifying at least 2,000 polymorphic or SNP loci in a single reaction volume. In some embodiments, the targeted amplification comprises amplifying at least 5,000 polymorphic or SNP loci in a single reaction volume. In some embodiments, the targeted amplification comprises amplifying at least 10,000 polymorphic or SNP loci in a single reaction volume.

In some embodiments, each primer pair is designed to amplify a target sequence of about 35 to 200 bp. In some embodiments, each primer pair is designed to amplify a target sequence of about 50 to 100 bp. In some embodiments, each primer pair is designed to amplify a target sequence of about 60 to 75 bp. In some embodiments, each primer pair is designed to amplify a target sequence of about 65 bp.

In some embodiments, the transplant recipient is a human subject. In some embodiments, the transplant recipient received an allograft. In some embodiments, the transplant recipient received a xenograft.

In some embodiments, the transplant is a human transplant. In some embodiments, the transplant is a pig transplant. In some embodiments, the transplant is from a non-human animal.

In some embodiments, the transplant is an organ transplant, tissue transplant, or cell transplant. In some embodiments, the transplant is a kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, or blood transfusion.

In some embodiments, the method further comprises determine the transplant rejection as antibody mediated transplant rejection, T-cell mediated transplant rejection, graft injury, viral infection, bacterial infection, or borderline rejection.

### WORKING EXAMPLES

### Example 1

This example is illustrative only, and a skilled artisan will appreciate that the invention disclosed herein can be practiced in a variety of other ways.

### Blood Samples

Male and female adult or young-adult patients receives a donor organ from related or unrelated living donors, or unrelated deceased donors. Time points of patient blood draw following transplantation surgery are either at the time of an allograft biopsy or at various pre-specified time intervals based on lab protocols. Typically, samples are biopsy-matched and blood are drawn at the time of clinical dysfunction and biopsy or at the time of protocol biopsy (at which time most patients do not have clinical dysfunction). In addition, some patients had serial post transplantation blood drawn. The selection of study samples was based on (a) adequate plasma being available, and (b) if the sample was associated with biopsy information. Among the full 300 sample cohort, 72.3% were drawn on the day of biopsy.

### Nucleic acid Measurement in Blood Samples

Nucleic acids such as RNA or DNA, and in particular cell-free DNA, mRNA, and microRNA is extracted from plasma samples using the QIAAMP™ Circulating Nucleic Acid Kit (Qiagen) and the LABCHIP™ NGS 5k kit (Perkin Elmer, Waltham, MA, USA) is used for quantification. Library preparation is performed using the Natera Library Prep kit as described in Abbosh et al, Nature 545:446-451 (2017), with a modification of 18 cycles of library amplification to plateau the libraries. Purified libraries are quantified using LABCHIP™ NGS 5k as described in Abbosh et al, Nature 545:446-451 (2017). Target enrichment is accomplished using massively multiplexed-PCR (mmPCR) using a modified version of described in Zimmermann et al., Prenat. Diagn. 32:1233-1241 (2012), with 13,392 single nucleotide polymorphisms (SNPs) targeted. Amplicons are then sequenced on an Illumina HiSeq 2500 RAPIDRUN®, 50 cycles single end, with 10-11 million reads per sample.

### Statistical Analyses of Nucleic Acids, dd-cfDNA and eGFR

In each sample, donor derived RNA and/or dd-cfDNA was measured and correlated with rejection status, and results are compared with eGFR. Where applicable, all statistical tests are two sided. Significance is set at p <0.05. Because the distribution of dd-cfDNA in patients is severely skewed among the groups, data are analyzed using a Kruskal-Wallis rank sum test followed by Dunn multiple comparison tests with Holm correction. eGFR (serum creatinine in mg/dL) is calculated as described previously for adult and pediatric patients. Briefly, eGFR=186×Serum Creatinine^{-1.154}×Age^{-0.203}×(1.210 if Black)×(0.742 if Female).

Removing contamination using Cas9/Cas12a or Cas13 CRISPR technology. The Cas9/Cas12a CRISPR system may be used to remove contaminants from the cDNA library. The Cas13 system may be used to remove contaminating RNA from the biological sample.

### Example 2

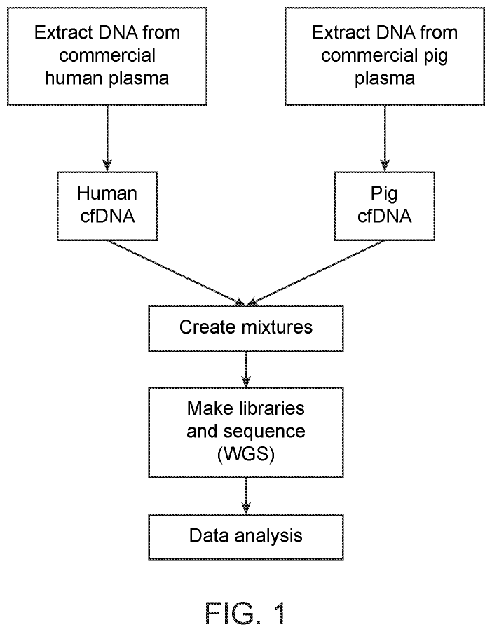

This example evaluated the xenotransplant monitoring test as disclosed herein. FIG. 1 shows an outline of one of the testing methods of detecting cell-free DNA from animal donor in human plasma sample.

First, cell-free DNA (cfDNA) was purified from human and pig plasma samples. Next, combinations of pig and human cell-free DNA were made. The combinations tested mixtures containing from 100% down to 0.01% pig cell-free DNA. Sequencing libraries were prepared from the combinations of pig and human cfDNA.

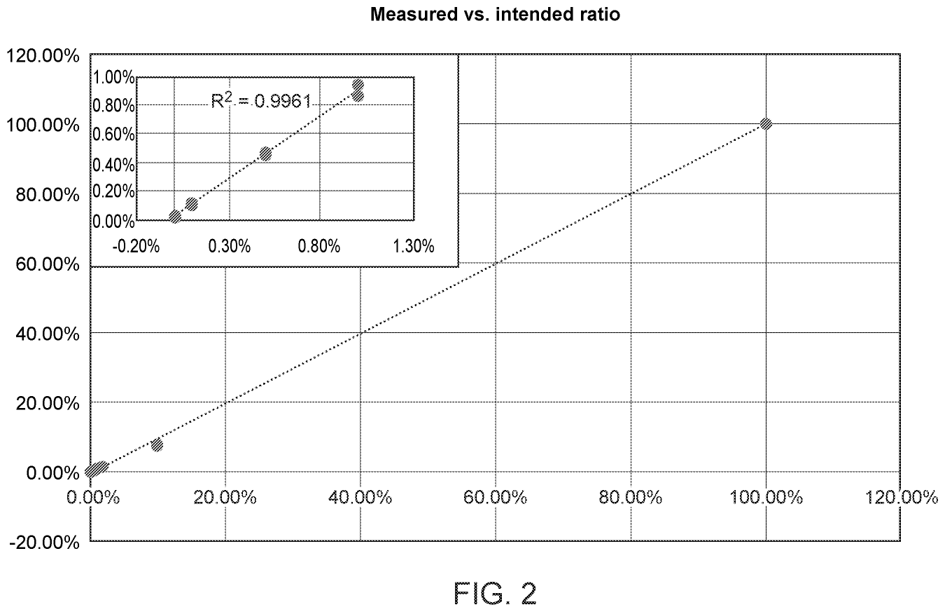

The ratio of pig/human cfDNA of each combination was determined by using whole genome sequencing of the prepared libraries, and the determined ratios of pig/human cfDNA were plotted against the actual ratio of each sample as shown in FIG. 2. Linear regression analysis showed that there was a linear relationship (R2=0.9961) between measured and actual ratios of pig/human cfDNA. Accordingly, this evaluation proved the concept of using whole genome sequencing of cfDNA extracted from plasma samples to measure amounts of cfDNA from a xenotransplant, which is useful for monitoring xenotransplant rejection.

What is claimed is:

1. A method of preparing a non-naturally-occurring composition of DNA derived from a blood, plasma, serum or urine sample of a xenotransplant recipient useful for determination of xenotransplant rejection, comprising:
(a) extracting cell-free DNA from the blood, plasma, serum or urine sample of the xenotransplant recipient, wherein the extracted cell-free DNA comprises donor-derived cell-free DNA and recipient-derived cell-free DNA;
(b) preparing a sequencing library from the extracted cell-free DNA, and sequencing the sequencing library by high-throughput sequencing to obtain sequencing reads and quantifying the total amount of animal donor-derived cell-free DNA based on the sequencing reads;
(c) determining whether the amount of donor-derived cell-free DNA from the xenotransplant or a function thereof exceeds a cutoff threshold indicating xenotransplant rejection or injury.

2. A method of preparing a non-naturally-occurring composition of amplified DNA derived from a blood, plasma, serum or urine sample of a xenotransplant recipient useful for determination of xenotransplant rejection, comprising:
(a) extracting cell-free DNA from the blood, plasma, serum or urine sample of the xenotransplant recipient, wherein the extracted cell-free DNA comprises donor-derived cell-free DNA from the xenotransplant and recipient-derived cell-free DNA;
(b) preparing a composition of amplified DNA by performing multiplex targeted amplification of the extracted cell-free DNA at 10-50,000 target loci in a single reaction volume to detect and quantify the amount of animal donor-derived cell-free DNA, wherein the target loci comprise a set of animal target loci and a set of human target loci;
(c) determining whether the amount of animal donor-derived cell-free DNA or a function thereof exceeds a cutoff threshold indicating xenotransplant rejection.

3. A method of preparing a non-naturally-occurring composition of amplified DNA derived from a blood, plasma, serum or urine sample of a xenotransplant recipient useful for determination of xenotransplant rejection, comprising: (a) extracting cell-free DNA from the blood, plasma, serum or urine sample of the transplant recipient, wherein the extracted cell-free DNA comprises donor-derived cell-free DNA and recipient-derived cell-free DNA;
(b) preparing a composition of amplified DNA by performing targeted amplification of the extracted cell-free DNA at 10-50,000 target loci in a single reaction volume, wherein the target loci comprise both human and animal target loci, and sequencing the amplified DNA by high-throughput sequencing to obtain sequencing reads and quantifying the amount of both donor-derived cell-free DNA and xenotransplant recipient-derived cell-free DNA based on the sequencing reads, wherein the human and animal loci are the same, and wherein the human and animal reads are distinguished based on an insert sequence;
(c) determining whether the fraction of animal donor-derived cell-free DNA or a function thereof exceeds a cutoff threshold indicating xenotransplant rejection.

4. A method of preparing a non-naturally-occurring composition of amplified DNA derived from a blood, plasma, serum or urine sample of a xenotransplant recipient useful for determination of xenotransplant rejection, comprising:
(a) extracting cell-free DNA from the blood, plasma, serum or urine sample of the xenotransplant recipient, wherein the extracted cell-free DNA comprises donor-derived cell-free DNA and recipient-derived cell-free DNA;
(b) preparing a composition of amplified DNA by performing targeted amplification of the extracted cell-free DNA at 10-50,000 target loci in a single reaction volume, wherein the target loci comprise one or more target loci indicating xenotransplant rejection; and
(c) determining an amount of the one or more target loci indicating xenotransplant rejection, and determining whether the amount of the one or more target loci indicating xenotransplant rejection or a function thereof exceeds a cutoff threshold indicating xenotransplant rejection.

5. The method of claims 1-4, wherein the composition of DNA comprises one or more target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection;
wherein xenotransplant rejection is determined by a combination of (i) the amount of the one or more target loci indicating xenotransplant rejection or a function thereof, and (ii) the total amount of animal donor-derived cell-free DNA or the fraction of animal donor-derived cell-free DNA.

6. A method of preparing a non-naturally-occurring composition of complimentary DNA (cDNA) from RNA extracted from a blood, plasma, serum or urine sample of a xenotransplant recipient useful for determination of xenotransplant rejection, comprising:
(a) extracting RNA from the blood, plasma, serum or urine sample of the xenotransplant recipient, wherein the extracted RNA comprises donor-derived RNA and recipient-derived RNA;
(b) preparing a sequencing library of cDNA from the extracted RNA, and sequencing the sequencing library of cDNA by high-throughput sequencing to obtain sequencing reads and quantifying the total amount of donor-derived RNA based on the sequencing reads;
(c) determining whether the total amount of donor-derived RNA from the xenotransplant or a function thereof exceeds a cutoff threshold indicating xenotransplant rejection.

7. A method of preparing a non-naturally-occurring composition of amplified complimentary DNA (cDNA) from RNA extracted from a blood, plasma, serum or urine sample of a xenotransplant recipient useful for determination of xenotransplant rejection, comprising:
(a) extracting RNA from the blood, plasma, serum or urine sample of the xenotransplant recipient, wherein the extracted RNA comprises donor-derived RNA from the xenotransplant and recipient-derived RNA;
(b) preparing a composition of amplified cDNA derived from the extracted RNA by performing multiplex targeted amplification of the cDNA at 10-50,000 target loci in a single reaction volume to detect and quantify the amount of donor-derived RNA, wherein the target loci comprise a set of animal target loci and a set of human target loci;
(c) determining whether the amount of donor-derived RNA target loci or a function thereof exceeds a cutoff threshold indicating xenotransplant rejection.

8. A method of preparing a non-naturally-occurring composition of amplified complimentary DNA (cDNA) from RNA extracted from a blood, plasma, serum or urine sample of a xenotransplant recipient useful for determination of xenotransplant rejection, comprising:
(a) extracting RNA from the blood, plasma, serum or urine sample of the xenotransplant recipient, wherein the extracted RNA comprises donor-derived RNA and recipient-derived RNA;
(b) preparing a composition of amplified cDNA derived from the extracted RNA by performing targeted amplification of the cDNA at 10-50,000 target loci in a single reaction volume, wherein the target loci comprise both human and target loci, and sequencing the amplified cDNA by high-throughput sequencing to obtain sequencing reads and quantifying the amount of both donor-derived RNA and xenotransplant recipient-derived RNA based on the sequencing reads, wherein the human and animal loci are the same, and wherein the human and animal reads are distinguished based on an insert sequence;
(c) determining whether the fraction of donor-derived RNA or a function thereof exceeds a cutoff threshold indicating xenotransplant rejection.

9. A method of preparing a non-naturally-occurring composition of amplified complimentary DNA (cDNA) from RNA extracted amplified complimentary DNA (cDNA) from RNA extracted from a blood, plasma, serum or urine sample of a xenotransplant recipient useful for determination of xenotransplant rejection, comprising:
(a) extracting RNA from the blood, plasma, serum or urine sample of the xenotransplant recipient, wherein the extracted RNA comprises donor-derived cell-free RNA and recipient-derived RNA;
(b) preparing a composition of amplified cDNA derived from the extracted RNA by performing targeted amplification of the cDNA at 10-50,000 target loci in a single reaction volume, wherein the target loci comprise one or more target loci indicating xenotransplant rejection; and
(c) determining an amount of the one or more target loci indicating xenotransplant rejection, and whether the amount of the one or more target loci indicating xenotransplant rejection or a function thereof exceeds a cutoff threshold indicating xenotransplant rejection.

10. The method of claims 6-9, wherein the composition comprises target loci indicating xenotransplant rejection; and wherein the determining step further comprises determining an amount of the one or more target loci indicating xenotransplant rejection, and determining whether the amount of the one or more target loci indicating xenotransplant rejection or a function thereof exceeds a cutoff threshold indicating xenotransplant rejection, wherein xenotransplant rejection is determined by a combination of the amount of the one or more target loci indicating xenotransplant rejection or a function thereof, and the total amount of animal donor-derived RNA or the fraction of animal donor-derived RNA.

11. A method of preparing a non-naturally-occurring composition of protein derived from a blood, plasma, scrum or urine sample of a xenotransplant recipient useful for determination of xenotransplant rejection, comprising:
(a) extracting protein from the blood, plasma, serum or urine sample of the xenotransplant recipient, wherein the protein comprises donor-derived protein from the xenotransplant and recipient-derived protein;
(b) detecting and quantifying an amount of donor-derived protein;
(c) determining whether the amount of protein donor-derived protein or a function thereof exceeds a cutoff threshold indicating xenotransplant rejection.

12. A method of preparing a non-naturally occurring composition of protein derived from a blood, plasma, serum or urine sample of a xenotransplant recipient useful for determination of xenotransplant rejection, comprising:
(a) extracting protein from the blood, plasma, serum or urine sample of the xenotransplant recipient, wherein the protein comprises donor-derived protein from the xenotransplant and recipient-derived protein;
(b) detecting and quantifying an amount of one or more target proteins, wherein the one or more target proteins comprise both recipient-derived proteins and donor-derived proteins, and wherein the one or more target proteins are indicative of xenotransplant rejection;
(c) determining whether the amount of the one or more target proteins or a function thereof exceeds a cutoff threshold indicating xenotransplant rejection.

13. A method of administrating immunosuppressive therapy in a xenotransplant recipient, comprising:
(a) measuring an amount of donor-derived protein according to claim 11; and
(b) titrating the dosage of an immunosuppressive therapy according to the amount of donor-derived protein or a function thereof.

14. A method of administrating immunosuppressive therapy in a xenotransplant recipient, comprising:
(a) measuring an amount of one or more target proteins according to claim 12; and (b) titrating the dosage of an immunosuppressive therapy according to the amount of the one or more target proteins or a function thereof.

15. The method of any of claims 13-14, further comprising repeating steps (a)-(b) longitudinally for the same xenotransplant recipient, and determining a longitudinal change in the amount donor-derived protein, donor-derived target proteins, or a function thereof, and a longitudinal change in the amount of donor-derived protein, target proteins, or a function thereof.

16. The method of claim 15, further comprising titrating the dosage of the immunosuppressive therapy according to the longitudinal change in the donor-derived protein, donor-derived target proteins, or a function thereof.

17. The method of any one of claims 11-16, wherein the protein is derived from extracellular vehicles (EVs) isolated from the blood, plasma, serum, or urine samples of a xenotransplant recipient, and comprises extracting protein from extracellular vesicles (EVs) isolated from the blood, plasma, serum or urine sample of the xenotransplant recipient.

18. A method of administrating immunosuppressive therapy in a xenotransplant recipient, comprising:
(a) measuring the amount of cell-free DNA in a blood, plasma, serum, or urine sample of the transplant recipient; and
(b) measuring the total amount of donor-derived cell-free DNA in a blood, plasma, serum, or urine sample of the transplant recipient; and
(c) titrating the dosage of an immunosuppressive therapy according to the amount of cell-free DNA or a function thereof and the amount of donor-derived cell-free DNA or a function thereof.

19. The method of claim 18, wherein the amount of donor-derived cell-free DNA is measured by:
extracting cell-free DNA from the blood, plasma, serum or urine sample of the transplant recipient, wherein the extracted cell-free DNA comprises donor-derived cell-free DNA and recipient-derived cell-free DNA;
performing targeted amplification of the extracted DNA at 200-50,000 target loci in a single reaction volume;
sequencing the amplified DNA by high-throughput sequencing to obtain sequencing reads and quantifying the amount of donor-derived cell-free DNA based on the sequencing reads.

20. The method of any of claims 18-19, further comprising repeating steps (a)-(b) longitudinally for the same transplant recipient, and determining a longitudinal change in the amount of cell-free DNA or a function thereof and a longitudinal change in the amount of donor-derived cell-free DNA or a function thereof.

21. The method of claim 20, further comprising titrating the dosage of the immunosuppressive therapy according to the longitudinal change in the total amount of cell-free DNA or a function thereof and the longitudinal change in the amount of donor-derived cell-free DNA or a function thereof.

22. A method of administrating immunosuppressive therapy in a xenotransplant recipient, comprising:
(a) measuring the amount of RNA in a blood, plasma, serum, or urine sample of the transplant recipient; and
(b) measuring the amount of donor-derived RNA in a blood, plasma, serum, or urine sample of the transplant recipient; and
(c) titrating the dosage of an immunosuppressive therapy according to the amount of cell-free DNA or a function thereof and the amount of donor-derived RNA or a function thereof.

23. The method of claim 20, the amount of donor-derived RNA is measured by:
extracting RNA from the blood, plasma, serum or urine sample of the transplant recipient, wherein the extracted RNA comprises donor-derived RNA and recipient-derived RNA;
preparing a composition of amplified complimentary DNA (cDNA) derived from the extracted RNA by performing multiplex targeted amplification of the cDNA at 200-50,000 animal target loci in a single reaction volume to detect and quantify the amount of animal donor-derived RNA;
sequencing the amplified cDNA by high-throughput sequencing to obtain sequencing reads and quantifying the amount of donor-derived RNA based on the sequencing reads.

24. The method of any of claims 22-23, further comprising repeating steps (a)-(b) longitudinally for the same transplant recipient, and determining a longitudinal change in the amount of RNA or a function thereof and a longitudinal change in the amount of donor-derived RNA or a function thereof.

25. The method of claim 24, further comprising titrating the dosage of the immunosuppressive therapy according to the longitudinal change in the total amount of RNA or a function thereof and the longitudinal change in the amount of donor-derived cell-free DNA or a function thereof.

26. The method of any one of claims 1-10 and 18-21, wherein an internal control is added to the sample.

27. The method of any one of claims 1-10 and 18-21, wherein the sequencing comprises shotgun whole genome sequencing.

28. The method of any one of the claims according to claims 1-10 and 18-25, wherein the amount of DNA or RNA is measured by quantitative PCR, real-time PCR, digital PCR, or sequencing.

29. The method of claim 28, wherein sequencing comprises next-generation whole genome sequencing.

30. The method of any one of the claims according to claims 1-10 and 18-25, wherein the amount of RNA or cell-free DNA is measured by using microarray.

31. The method of any one of the claims according to claims 1-10 and 18-25, wherein the amount of donor-derived RNA or cell-free DNA is determined by using ratiometric and/or machine learning-artificial intelligence comparisons at a single or a plurality of time points.

32. The method of any one of the claims according to claims 1-10 and 18-25, wherein the amount of RNA or cell-free DNA is measured by using molecular barcodes and microscopic imaging.

33. The method of any one of the claims according to claims 1-10 and 18-32, wherein the target loci comprise single nucleotide polymorphisms (SNPs).

34. The method of any one of the claims according to claims 1-10 and 18-33, wherein the cutoff threshold is an estimated percentage of donor-derived cell-free DNA or RNA out of total cell-free DNA or RNA or a function thereof.

35. The method of any one of the claims according to claims 1-10 and 18-34, wherein the cutoff threshold is proportional to an absolute donor-derived cell-free DNA concentration or RNA concentration. 108

36. The method of any one of the claims according to claims 1-10 and 18-34, wherein step (b) comprises amplification of at least 2, at least 5, at least 10, at least 20, at least 30, at least 50, at least 100 target loci, from 2-10, 200-100, 50-500, or 50-2000 target loci, using at least at least 2, at least 5, at least 10, at least 20, at least 30, at least 50, at least 100 target RNA molecules, from 2-10, 200-100, 50-500, or 50-2000 pairs of forward and reverse PCR primers.

37. The method of any one of the claims according to claims 1-10 and 18-34, wherein step (b) comprises multiplex amplification of at least 100, at least 500, at least 1000, at least 2000 target loci, from 10-1000, 100-10000, 50-50000, or 500-20000 target loci, using at least 100,at least 500, at least 1000, at least 2000, from 10-1000, 100-10000, 50-50000, 500-20000 pairs of forward and reverse PCR primers.

38. The method of any one of claims 6-10, and 22-37, wherein the RNA is cell-free RNA.

39. The method of claim 37, wherein the cell-free RNA is derived from exosomes or microvesicles.

40. The method of any one of claims 6-10, and 22-39, wherein the RNA is small messenger RNA (mRNA).

41. The method of any one of claims 6-10, and 22-39, wherein the RNA is small noncoding RNA (sncRNA).

42. The method of claim 39, wherein the sncRNA comprises micro RNA (miRNA), piwi-interacting RNA (piRNA), small nucleolar RNA (snoRNA), small nuclear RNA (snRNA), or miscellaneous RNA (miscRNA).

43. The method of any one of claims 6-10 and 22-42, further comprising utilizing CRISPR-Cas to target and deplete contaminating or overabundant nucleic acid species in the sample, thereby increasing the fraction of desired reads that map to a target loci of interest per sample and the sample throughput per sequencing run 44. The method of claim 43, wherein the sample comprises whole blood or hemolysis tainted blood, serum or plasma samples, and wherein a plurality of guide RNAs are used to target a plurality of loci in the same reaction to deplete contaminating or overabundant nucleic acid, thereby increasing a detection rate of the target loci.

45. The method of claim 43, wherein the contaminating or overabundant nucleic acid species comprise hemoglobin mRNA, tRNA, and rRNA, and miR-451, miR-144, and miR-486.

46. The method of claims 43-45, further comprising depleting adaptor dimers, primer dimers, unwanted ligation products from the composition of amplified nucleic acids comprising target loci, thereby increasing the fraction of desired reads that map to a target loci of interest per sample and the sample throughput per sequencing run.

47. The method of claims 43-46, wherein a CRISPR-Cas system comprising Cas9 or Cas 12 is utilized to remove nucleic acid species after reverse transcription of RNA and before multiplex amplification.

48. The method of claims 43-47, wherein a CRISPR-Cas system comprising Cas9 or Cas12 is utilized to remove nucleic acid species after 1-10 cycles of multiplex amplification of the complementary DNA.

49. The method of claims 43-48, wherein the contaminating or overabundant nucleic acid species are RNA, and wherein a CRISPR-Cas system comprising Cas13 is used to remove the contaminating or overabundant RNA species from the sample.

50. The method of any one of the preceding claims, wherein the xenotransplant recipient is a human subject.

51. The method of any one of the preceding claims, wherein the xenotransplant recipient has received one or more xenotransplanted organs selected from pancreas, kidney, liver, heart, lung, intestinal, thymus, and uterus.

52. The method of any one of the preceding claims, wherein the sample is obtained from the xenotransplant recipient less than 18 months post-transplantation.

53. The method of any one of the preceding claims, wherein the rejection risk for the xenotransplant recipient is determined using logistic regression, random forest, or decision tree machine learning analysis.

54. The method of claim 53, wherein the logistic regression, random forest, or decision tree machine learning analysis further incorporates one or more parameters selected from time post-transplantation, age of xenotransplant recipient and/or xenotransplant donor, gender of xenotransplant recipient and/or xenotransplant donor.

55. The method of any one of the preceding claims, wherein the xenotransplant is from a pig, a primate, a baboon, a cow, or a dog.

56. The method of any one of the preceding claims, wherein the xenotransplant is from a pig.

57. The method of any one of claims 1-10 and 18-56, wherein the cell-free DNA or RNA is derived from extracellular vehicles (EVs) isolated from the blood, plasma, serum, or urine samples of a xenotransplant recipient.
